(19) United States
(12) Patent Application Publication
(10) Pub. No.: US 2010/0004306 A1
(43) Pub. Date: Jan. 7, 2010

(54) PIGF-1 Assay and kits and components thereof

(75) Inventors: Evelyn Mary McKeegan, Lake Forest, IL (US); Saul A. Datwyler, Evanston, IL (US); David J. Hawksworth, Lake Villa, IL (US); Don M. Laird, Mundelein, IL (US); Dominick L. Pucci, Libertyville, IL (US); David C. Sogin, Highland Park, IL (US); Joan D. Tyner, Beach Park, IL (US); Robert N. Ziemann, Lindenhurst, IL (US); Peter James Ansell, Grayslake, IL (US); Ke K. Zhang, Glenview, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/486,671

(22) Filed: Jun. 17, 2009

Related U.S. Application Data
(63) Continuation-in-part of application No. 12/485,114, filed on Jun. 16, 2009.
(60) Provisional application No. 61/110,063, filed on Oct. 31, 2008.
(60) Provisional application No. 61/073,624, filed on Jun. 18, 2008.
(60) Provisional application No. 61/089,172, filed on Aug. 15, 2008.

(51) Int. Cl. A61K 31/416; G01N 33/566; A61P 35/00

(52) U.S. Cl. 514/406; 436/501

(57) ABSTRACT

The present disclosure relates to, among other things, methods for determining whether a subject receiving treatment with a drug has obtained an efficacious blood level of the drug. Moreover, the present disclosure also relates to methods of determining whether a subject predisposed to or suffering from a disease will benefit from treatment with a drug, and the response of a subject receiving treatment (e.g., such as for cancer) by monitoring biomarkers of angiogenesis. In particular, the disclosure relates to PlGF-1 companion diagnostic methods and products.

### RELATED APPLICATION INFORMATION

This application claims the benefit of U.S. application Ser. No. 12/485,114, filed on Jun. 16, 2009, the benefit of U.S. Application Ser. No. 61/110,063 filed Oct. 31, 2008, U.S. Application No. 61/073,624 filed on Jun. 18, 2008 and U.S. Ser. No. 61/089,172 filed on Aug. 15, 2008, the contents of each of which are herein incorporated by reference.

### REFERENCE TO JOINT RESEARCH AGREEMENT

Contents of this disclosure are under a joint research agreement entered into by and between Genentech and Abbott Laboratories on Jun. 22, 2007, directed to N-[4-(3-amino-1H-indazol-4-yl)phenyl]-N′-(2-fluoro-5-methylphenyl)urea (also known as ABT-869).

### TECHNICAL FIELD

The present disclosure relates, among other things, to methods for determining whether a subject receiving treatment with a drug (e.g., such as for cancer) has obtained an efficacious blood level of the drug by monitoring the levels of biomarkers of angiogenesis. Moreover, the present disclosure also relates to methods of determining whether a subject predisposed to or suffering from a disease will benefit from treatment with a drug, and the response of a subject receiving treatment (e.g., such as for cancer) by monitoring biomarkers of angiogenesis. In particular, the disclosure relates to PlGF-1 companion diagnostic methods and products.

### BACKGROUND

Neoangiogenesis is crucial for tumor growth. Specifically, neoangiogenesis is a complex process that involves the imbalance of antiangiogenic and proangiogenic molecules such as vascular endothelial growth factors secreted from tumor cells, macrophages and stromal cells that interact with vascular endothelial growth factor receptor (VEGFR) 1 and 2 to activate endothelial cells from existing microvasculature and circulating endothelial progenitor cells (See, Dvorak, H F, J Clin Oncol., 20:4368-4380 (2002)). Tumor vasculature is disorganized and more leaky than normal vasculature, thereby resulting in increased permeability, raised interstitial pressure and poorer chemotherapy distribution into the tumor (See, Ferrara, N., VEGF Oncology, 69:S11-S16, (2005) (suppl 3)). Platelet derived growth factor through binding to its receptor (PDGFR) activates tumor growth and enhances angiogenesis by facilitating pericyte coverage of new microvessels (See, Carmeliet, P., Nat Med., 9:653-660 (2003); Benjamin L E, et al., J Clin Invest., 103:159-165 (1999)), while VEGFR-3 contributes to the process by facilitating lymphangiogenesis, and may be potentially important for tumor metastases (Sundar, S S, et al., J Clin Oncol., 25:4298-4307 (2007)). Overactivation of these pathways promotes growth, tumor survival and metastasis of cancer cells (See, Blume-Jensen P, et al., Nature, 411:355-365 (2001)). In view thereof, VEGFR-1,-2,-3 and PDGFRs are important targets for inhibiting angiogenesis and lymphangiogenesis as well as the restoration of normalized vasculature. It is believed that the combined inhibition of VEGFR-1,-2,-3 and PDGFRs might disrupt tumor growth, survival and metastases more effectively than the specific inhibition of each receptor alone. Sorafenib and sunitinib are examples of small molecule receptor tyrosine kinase (RTK) inhibitors that are used for treating metastatic renal cell carcinoma (See, Batchelor, T T, et al., Cancer Cell, 11:83-95 (2007); Escudier, B, et al., N Engl J Med 356:125-134 (2007)), in which the loss of the von Hippel Lindau gene results in aberrant activation of hypoxic inducible factor complex and consequent VEGF and PDGF over-expression (Motzer, R J, et al., N Engl J Med., 356:115-124 (2007)).

A number of biomarkers associated with specific types of diseases are known. In this era of personalized medicine (involving the use of new methods of analysis and bioinformatics to better address and manage a patient's disease or predisposition to disease) and pharmacogenomics (combining the science of drugs and genomics) have promoted the use and interrogation of so-called “companion diagnostics”, which are diagnostic products intended for use in conjunction with a therapeutic product to better inform treatment selection, initiation, dose customization, or avoidance. Establishing a correlation between biomarkers and the pharmacological effect and biologic activity of a drug or pharmaceutical composition via assessment of plasma levels of these biomarkers thus can be used to individualize patient prognostication and treatment strategies. In this regard, a number of biomarkers are associated with angiogenesis. These include, but are not limited to, VEGF-A, soluble VEGFR-1 (sVEGFR-1, also known as sFlt-1), soluble VEGFR-2 (sVEGFR-2 also known as sKDR), soluble VEGFR-3 (sVEGFR-3), placenta growth factor (PlGF, a member of the VEGF family of growth factors and a specific ligand of VEGFR-1), erythropoietin, etc.

Thereupon, there is a need in the art for methods for monitoring or determining whether a subject receiving treatment with a drug (e.g., such as for cancer) has obtained an efficacious blood level of the drug by monitoring biomarkers of angiogenesis, particularly PlGF. Specifically, there is also a need in the art for methods of identifying whether a subject, after receiving just a single dose of a drug (e.g., such as for cancer) would benefit from continued treatment with said drug. Moreover, there is also a need in the art for methods of monitoring the response of a subject receiving treatment for a tumor (e.g., such as for cancer) by monitoring biomarkers of angiogenesis, particularly PlGF, and especially PlGF-1. Still further, there is also a need in the art for methods of using pharmacodynamic based dosing information to bring subjects into the necessary drug exposure range (e.g., area under the curve (“AUC”)) that is been associated or correlated with the efficacy of a specific drug. These and other objects of the disclosure will be apparent from the description following herein.

### SUMMARY

In one embodiment, the present disclosure provides a method of monitoring whether a subject being administered a drug has obtained an efficacious blood level of the drug in order to optimize dosing or scheduling, the method comprising the steps of:

(a) contacting a test sample obtained from a subject being administered N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea with a first capture antibody that binds to human PlGF-1 or a human PlGF-1 fragment to form a first capture antibody-human PlGF-1 complex,

(b) contacting the first capture antibody-human PlGF-1 complex with a second antibody that binds to human PlGF-1 or human PlGF-1 fragment and that has been conjugated to a detectable label (“detection antibody”) to form a second capture antibody-human PlGF-1 detection complex;

(c) determining the amount of the second capture antibody-human PlGF-1 detection complex formed in step (b) by detecting the detectable label, wherein the amount of the second complex formed is the amount of human PlGF-1 or human PlGF-1 fragment contained in the test sample; and

(d) comparing the amount of human PlGF-1 or human PlGF-1 fragment in the test sample determined in step (c) with a predetermined level, wherein if the concentration of human PlGF-1 or human PlGF-1 fragment determined in step (c) is lower than the predetermined level, then the subject is considered not to be receiving an efficacious amount of N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea and further wherein, if the concentration of human PlGF-1 or human PlGF-1 fragment determined in step (c) is the same as or higher than the predetermined level, then the subject is considered to be receiving an efficacious amount of N-[4-(3-amino-1H-indazol-4-yl)phenyl]-N′-(2-fluoro-5-methylphenyl)urea or analog of N-[4-(3-amino-1H-indazol-4-yl)phenyl]-N′-(2-fluoro-5-methylphenyl)urea.

In one aspect of such a method, the capture antibody is monoclonal antibody 264 and the detection antibody is polyclonal antibody pB264. Optionally, the predetermined level is about 30 picograms per milliliter at about 24 hours after the subject first receives treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea. Alternately, optionally, the predetermined level is about 40 picograms per milliliter to about 75 picograms per milliliter at when the patient has achieved steady state concentrations following treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea.

In another aspect of such a method, the capture antibody is monoclonal antibody 826 and the detection antibody is monoclonal antibody 255. In one aspect of this method, when the capture antibody is a monoclonal antibody and the detection antibody is a monoclonal antibody and the concentration of human PlGF-1 or human PlGF-1 fragment determined in step (c) is increased by about 60 picograms per milliliter to about 150 picograms per milliliter when compared to the predetermined level at the steady state about either 8 or 15 days after the subject first receives treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea.

The methods as described herein can be employed wherein the subject is being treated for cancer selected from the group consisting of lung cancer, breast cancer, stomach cancer, bladder cancer, colon cancer, pancreatic cancer, ovarian cancer, prostate cancer, renal cancer, hepatocellular cancer, rectal cancer, hematopoietic malignancies, glioblastoma and infantile hemangioma, among others. In one embodiment, the method is adapted for use in an automated system or semi-automated system.

In one aspect of the method, when the capture antibody is a monoclonal antibody and the detection antibody is a polyclonal antibody, the dose or schedule for treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea can be adjusted to place the patient in the range of about 40 picograms per milliliter to about 75 picograms per milliliter based on the comparison in step (d).

In another aspect of the method, when the capture antibody is a monoclonal antibody and the detection antibody is a monoclonal antibody, the dose or schedule for treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea can be adjusted to place the patient in the range of about 60 picograms per milliliter to about 150 picograms per milliliter based on the comparison in step (d).

Accordingly provided by the description herein is a method of monitoring a response of a subject receiving treatment for cancer with an anti-cancer drug, the method comprising the steps of:

(a) contacting a test sample obtained from a subject receiving treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea with a first capture antibody that binds to human PlGF-1 or human PlGF-1 fragment to form a first capture antibody-human PlGF-1 complex;

(b) contacting the first capture antibody-human PlGF-1 complex with a second antibody that binds to human PlGF-1 and that has been conjugated to a detectable label (“detection antibody”) to form a second capture antibody-human PlGF-1 detection complex;

(c) determining the amount of the second capture antibody-human PlGF-1 detection complex formed in step (b) by detecting the detectable label, wherein the amount of the second complex formed is the amount of human PlGF-1 or human PlGF-1 contained in the test sample; and

(d) comparing the amount of human PlGF-1 or human PlGF-1 in the test sample determined in step (c) with a predetermined level, wherein if the concentration of human PlGF-1 or human PlGF-1 fragment determined in step (c) is lower than the predetermined level, then the subject is considered not to be responding to treatment with the N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea and treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea is discontinued and the subject is administered an anti-cancer drug other than N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea and further wherein, if the concentration of human PlGF-1 or human PlGF-1 fragment determined in step (c) is the same as or higher than the predetermined level, then the subject is considered to be responding to treatment with the N-[4-(3-amino-1H-indazol-4-yl)phenyl]-N′-(2-fluoro-5-methylphenyl)urea or analog of N-[4-(3-amino-1H-indazol-4-yl)phenyl]-N′-(2-fluoro-5-methylphenyl)urea.

In one aspect of this method, optionally, the capture antibody is monoclonal antibody 264 and the detection antibody is polyclonal antibody pB264. In another aspect of this method, optionally, the capture antibody is monoclonal antibody 826 and the detection antibody is monoclonal antibody 255. The method can be employed where the cancer is selected from the group consisting of lung cancer, breast cancer, stomach cancer, bladder cancer, colon cancer, pancreatic cancer, ovarian cancer, prostate cancer, renal cancer, hepatocellular cancer, rectal cancer, rectal cancer, hematopoietic malignancies, glioblastoma and infantile hemangioma, among others. In one embodiment, the method is adapted for use in an automated system or semi-automated system.

Further provided herein is another method that provides for determining whether a subject who is predisposed to a disease or who is suffering from a disease will benefit from receiving treatment with a drug. Optionally the method comprises the steps of:

(a) contacting a test sample obtained from a subject predisposed to a disease or suffering from at least one disease and administered N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea with a first capture antibody that binds to human PlGF-1 or human PlGF-1 fragment to form a first capture antibody-human PlGF-1 complex;

(b) contacting the first capture antibody-human PlGF-1 complex with a second antibody that binds to human PlGF-1 and that has been conjugated to a detectable label (“detection antibody”) to form a second capture antibody-human PlGF-1 detection complex;

(c) determining the amount of the second capture antibody-human PlGF-1 detection complex formed in step (b) by detecting the detectable label, wherein the amount of the second complex formed is the amount of human PlGF-1 or human PlGF-1 contained in the test sample; and

(d) comparing the amount of human PlGF-1 or human PlGF-1 in the test sample determined in step (c) with a predetermined level, wherein if the concentration of human PlGF-1 or human PlGF-1 fragment determined in step (c) is lower than the predetermined level, then a determination is made that the subject will not benefit from receiving further or continued treatment with the N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea and further wherein, if the concentration of human PlGF-1 or human PlGF-1 fragment determined in step (c) is the same as or higher than the predetermined level, then a determination is made that the subject will benefit from receiving further or continued treatment with the N-[4-(3-amino-1H-indazol-4-yl)phenyl]-N′-(2-fluoro-5-methylphenyl)urea or analog of N-[4-(3-amino-1H-indazol-4-yl)phenyl]-N′-(2-fluoro-5-methylphenyl)urea.

In one aspect of this method, optionally, the capture antibody is monoclonal antibody 264 and the detection antibody is polyclonal antibody pB264. In another aspect of this method, optionally, the capture antibody is monoclonal antibody 826 and the detection antibody is monoclonal antibody 255. In yet another aspect, optionally at least one disease is cancer selected from the group consisting of lung cancer, breast cancer, stomach cancer, bladder cancer, colon cancer, pancreatic cancer, ovarian cancer, prostate cancer, renal cancer, hepatocellular cancer, rectal cancer, rectal cancer, hematopoietic malignancies, glioblastoma and infantile hemangioma. In one embodiment, the method is adapted for use in an automated system or semi-automated system.

Also provided herein is a method of predicting the likelihood of response of a subject to treatment, wherein the method comprises the steps of:

(a) obtaining a first test sample prior to initiation of treatment obtained from a subject suffering from a disease and being administered N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea;

(b) contacting the first test sample with a first capture antibody that binds to human PlGF-1 or a human PlGF-1 fragment to form a first capture antibody-human PlGF-1 complex,

(c) contacting the first capture antibody-human PlGF-1 complex with a second antibody that binds to human PlGF-1 or human PlGF-1 fragment and that has been conjugated to a detectable label (“detection antibody”) to form a second capture antibody-human PlGF-1 detection complex;

(d) determining the amount of the second capture antibody-human PlGF-1 detection complex formed in step (b) by detecting the detectable label, wherein the amount of the second complex formed is the concentration of human PlGF-1 or human PlGF-1 fragment contained in the first test sample;

(e) obtaining a second test sample obtained from the subject suffering from the disease and being administered N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea at a period in time after obtaining the first test sample in step (a);

(f) contacting the second test sample with a first capture antibody that binds to human PlGF-1 or a human PlGF-1 fragment to form a first capture antibody-human PlGF-1 complex;

(g) contacting the first capture antibody-human PlGF-1 complex with a second antibody that binds to human PlGF-1 or human PlGF-1 fragment and that has been conjugated to a detectable label (“detection antibody”) to form a second capture antibody-human PlGF-1 detection complex;

(h) determining the amount of the second capture antibody-human PlGF-1 detection complex formed in step (g) by detecting the detectable label, wherein the amount of the second complex formed is the concentration of human PlGF-1 or human PlGF-1 fragment contained in the second test sample; and

(i) comparing the concentration of human PlGF-1 or human PlGF-1 fragment in step (d) with the concentration of human PlGF-1 or human PlGF-1 fragment in step (h), wherein if the concentration of human PlGF-1 or human PlGF-1 fragment determined in step (h) has increased when compared to the concentration of human PlGF-1 or human PlGF-1 fragment determined in step (d), then the patients with higher levels of PlGF-1 or human PlGF-1 fragment are more likely to respond if the concentration of human PlGF-1 or human PlGF-1 fragment determined in step (h) is unchanged or higher when compared to the concentration of human PlGF-1 or human PlGF-1 fragment in step (d).

In one aspect of this method, the period of time between obtaining the first test sample and the second test sample is a time period selected from the group consisting of about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years, and about 10.0 years. Optionally, the capture antibody is monoclonal antibody 264 and the detection antibody is polyclonal antibody pB264. Alternatively, optionally, the capture antibody is monoclonal antibody 826 and the detection antibody is monoclonal antibody 255. Further, optionally, the at least one disease is cancer selected from the group consisting of lung cancer, breast cancer, stomach cancer, bladder cancer, colon cancer, pancreatic cancer, ovarian cancer, prostate cancer, renal cancer, hepatocellular cancer, rectal cancer, rectal cancer, hematopoietic malignancies, glioblastoma and infantile hemangioma. Further optionally, in one aspect, the method is adapted for use in an automated system or semi-automated system.

In still yet another embodiment, the present invention relates to a method of treating a subject suffering from at least one cancer selected from the group consisting of lung cancer, breast cancer, stomach cancer, bladder cancer, colon cancer, pancreatic cancer, ovarian cancer, prostate cancer, renal cancer, hepatocellular cancer, rectal cancer, hematopoietic malignancies, glioblastoma and infantile hemangioma. Such a method comprises the steps of:

(a) obtaining a test sample from the subject suffering from cancer and who is receiving treatment with a predetermined amount of N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea;

(b) contacting the test sample with a first capture antibody that binds to human PlGF-1 or a human PlGF-1 fragment to form a first capture antibody-human PlGF-1 complex,

(c) contacting said first capture antibody-human PlGF-1 complex with a second antibody that binds to human PlGF-1 or human PlGF-1 fragment and that has been conjugated to a detectable label (“detection antibody”) to form a second capture antibody-human PlGF-1 detection complex;

(d) determining the amount of the second capture antibody-human PlGF-1 detection complex formed in step (c) by detecting the detectable label, wherein the amount of the second complex formed is the amount of human PlGF-1 or human PlGF-1 fragment contained in the test sample;

(e) comparing the amount of human PlGF-1 or human PlGF-1 fragment in the test sample determined in step (d) with a predetermined level; and

(f) treating a subject having a concentration of human PlGF-1 or human PlGF-1 fragment determined in step (d) that is lower than the predetermined level with: (i) an adjusted amount of N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea that is higher than the predetermined amount of N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea recited in step (a); (ii) a drug other N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea; or (iii) combinations of (i) and (ii).

In the above method, the capture antibody is monoclonal antibody 264 and the detection antibody is polyclonal antibody pB264.

Moreover, in the above method, optionally, the predetermined level when the capture antibody is a monoclonal antibody and the detection antibody is a polyclonal antibody assay is about 30 picograms per milliliter at about 24 hours after the subject first receives treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea. Still further optionally, in the above method, the predetermined level when the capture antibody is a monoclonal antibody and the detection antibody is a polyclonal antibody assay is about 40 picograms per milliliter to about 75 picograms per milliliter at about 15 days after the subject first receives treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea.

Alternatively, in the above method, the capture antibody is monoclonal antibody 826 and the detection antibody is monoclonal antibody 255.

Still further, in the above method, optionally, when the capture antibody is a monoclonal antibody and the detection antibody is a monoclonal antibody assay, the predetermined level is In one aspect of this method, when the capture antibody is a monoclonal antibody and the detection antibody is a monoclonal antibody and the concentration of human PlGF-1 or human PlGF-1 fragment determined in step (e) is increased by about 60 picograms per milliliter to about 150 picograms per milliliter when compared to the predetermined level at the steady state about either 8 or 15 days after the subject first receives treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea.

In still another embodiment, provided herein is a kit for use in determining whether a subject receiving treatment with a drug has obtained an efficacious blood level of the drug, wherein the drug is N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, the kit comprising:

(a) at least antibody selected from the group consisting of: monoclonal antibody 264, polyclonal antibody pB264 and combinations thereof; and

(b) instructions for using the kit.

Further provided is a kit for use in determining whether a subject receiving treatment with a drug has obtained an efficacious blood level of the drug, wherein the drug is N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, the kit comprising:

(a) at least antibody selected from the group consisting of: monoclonal antibody 826, monoclonal antibody 255 and combinations thereof; and

(b) instructions for using the kit

Further provided is a kit for use in monitoring a response of a subject receiving treatment for cancer with an anti-cancer drug, wherein the drug is N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, the kit comprising:

(a) at least antibody selected from the group consisting of: monoclonal antibody 264, polyclonal antibody pB264 and combinations thereof; and

(b) instructions for using the kit.

Still yet further provide is a kit for use in monitoring a response of a subject receiving treatment for cancer with an anti-cancer drug, wherein the drug is N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, the kit comprising:

(a) at least antibody selected from the group consisting of: monoclonal antibody 826, monoclonal antibody 255 and combinations thereof; and

(c) instructions for using the kit.

In yet another embodiment, provided herein is a kit for use in determining whether a subject who is predisposed to a disease or who is suffering from a disease will respond to treatment with a drug, wherein the drug is N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, the kit comprising:

(a) at least antibody selected from the group consisting of: monoclonal antibody 264, polyclonal antibody pB264 and combinations thereof; and

(b) instructions for using the kit.

In still yet another embodiment, provided herein is a kit for use in determining whether a subject who is predisposed to a disease or who is suffering from a disease will respond to treatment with a drug, wherein the drug is N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, the kit comprising:

(a) at least antibody selected from the group consisting of: monoclonal antibody 826, monoclonal antibody 255 and combinations thereof; and

(b) instructions for using the kit.

Further provided herein is a kit for use in monitoring progression of disease in a subject being administered with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, the kit comprising:

(a) at least antibody selected from the group consisting of: monoclonal antibody 264, polyclonal antibody pB264 and combinations thereof; and

(b) instructions for using the kit.

Still further provided herein is a kit for use in monitoring progression of disease in a subject being administered with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, the kit comprising:

(a) at least antibody selected from the group consisting of: monoclonal antibody 826, monoclonal antibody 255 and combinations thereof; and

(b) instructions for using the kit.

### DESCRIPTION OF THE FIGURES

FIG. 1 shows the dose responsive increase in human PlGF-1 at various time points as described in Example 1. In this Figure, -♦- is 0.1 mg/kg (N=11), -▪- is 0.25 mg/kg (N=11), -▴- is 0.3 mg/kg (N=3), and -*- is all patients (N=30).

FIG. 2 shows the optimal human PlGF-1 threshold which stratified patients for time on therapy (indicative of therapeutic benefit) as described in Example 1. Groups are shown for the 24 hour time point after initiation of treatment with ABT-869 as all patients (shown as the dotted ( . . . ) line (N=31) (with the Median TTP equal to 133)), greater than 30 pg/mL (shown as the dashed and dotted [symbol] line (N=20) (with the Median TTP equal to 144)) or less than 30 pg/mL (shown as the thick solid [symbol] line (N=11) with the Median TTP equal to 82)). Clear differences in the time to progression for each group are shown.

FIG. 3 shows the optimal human PlGF-1 threshold which stratified patients for time on therapy (indicative of therapeutic benefit) as described in Example 1. Groups are shown at the steady state time point after initiation of treatment with ABT-869 as either all patients (shown as the dotted ( . . . ) line (N=30) (with the Median TTP equal to 133)), greater than 40 pg/mL (shown as the dashed and dotted [symbol] line (N=17) (with the Median TTP equal to 328)) or less than 40 pg/mL (shown as the thick solid [symbol] line (N=13) with the Median TTP equal to 96)). Clear differences in the time to progression for each group are shown.

FIG. 4 shows the human PlGF-1 time course over a variety of time points as described in Example 1.

### DETAILED DESCRIPTION

It is known in the art that human PlGF-1 expression in tumor tissue correlates with poor prognosis and survival in subjects suffering from colorectal cancer (See, Wei, et al., Gut, 54:666-672 (2005)). Thus, one skilled in the art might anticipate that an effective therapy capable of reducing disease burden, causing remission or extending progression free survival, would result in decreasing plasma concentrations of human PlGF-1. Unexpectedly, the inventors of the present disclosure discovered that increasing human PlGF-1 concentrations in subjects suffering from a disease, such as cancer, can serve as a marker associated with effective therapy and improved time to progression, particularly in subjects suffering from cancer.

Thus, the present disclosure relates, among other things, to a human PlGF-1 or human PlGF-1 fragment companion diagnostic assay that can be used in concert with subjects receiving treatment with ABT-869 or an analog of ABT-869. More specifically, the present disclosure relates to methods for determining whether a subject receiving treatment with ABT-869 or an analog of ABT-869 has obtained an efficacious blood level of drug by monitoring the levels of PlGF-1 or human PlGF-1 fragment in said subject during the course of treatment. Moreover, the present disclosure also relates to methods of determining whether a subject predisposed to or suffering from a disease will benefit from treatment with a drug, and the response of a subject receiving treatment (e.g., such as for cancer) by monitoring biomarkers of angiogenesis. In particular, the disclosure relates to a PlGF-1 companion diagnostic methods and products. Optionally, the methods described herein are adapted for use on automated or semi-automated systems.

Finally, the present disclosure relates to methods of treating subjects suffering from one or more types of cancer or one or more types of autoimmune diseases.

### A. Definitions

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

a) ABT-869

As used herein, the term “ABT-869” refers to N-[4-(3-amino-1H-indazol-4-yl)phenyl]-N′-(2-fluoro-5-methylphenyl)urea which is an ATP-competitive receptor tyrosine kinase inhibitor that has potent inhibitory activity against VEGFR 1 (IC₅₀ 30 nM), 2 (IC₅₀ 8.5 nM), 3 (IC₅₀ 40 nM), PDGFRβ (IC₅₀ 25 nM), CSF-1R (IC₅₀ 5.3 nM) and Flt-3 kinase (IC₅₀ 9.5 nM) in kinase enzyme assays. ABT-869, methods for making ABT-869 (See, Example 5), types of formulations containing ABT-869 (e.g., capsules or tablets; powders or granules; solutions or suspensions in aqueous or non-aqueous liquids; edible foams or whips; or oil-in-water liquid emulsions or water-in-oil emulsions), routes of administration (e.g., oral (including buccal or sublingual), rectal, nasal, topical (including buccal, sublingual, or transdermal), vaginal, or parenteral (including subcutaneous, intramuscular, intravenous, or intradermal) route) are all described in U.S. Pat. No. 7,297,709, the contents of which are herein incorporated by reference.

b) Analogs of ABT-869 or ABT-869 Analogs

As used herein, the phrases “analogs of ABT-869” or “ABT-869 analogs” as used interchangeably herein refers pharmacologically active analogs, including, but not limited, to salts, esters, amides, prodrugs, conjugates, active metabolites, and other such derivatives, analogs, and related compounds of ABT-869. Exemplary analogs of ABT-869 include, but are not limited to, compounds having the below structural formulas (I-XX):

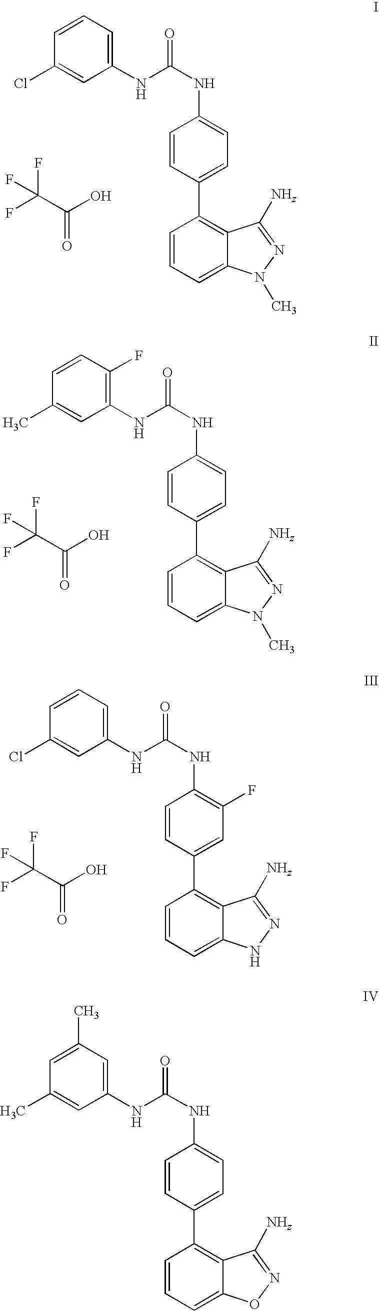

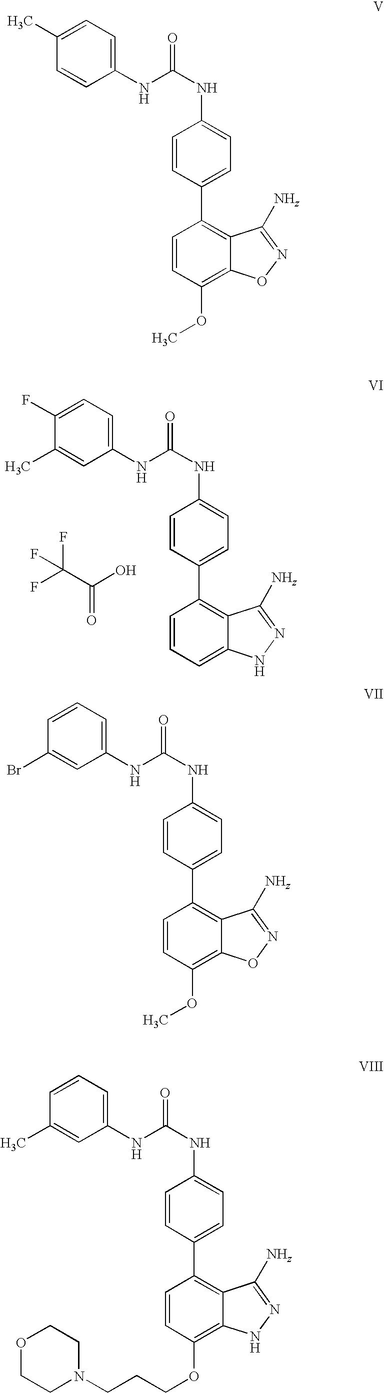

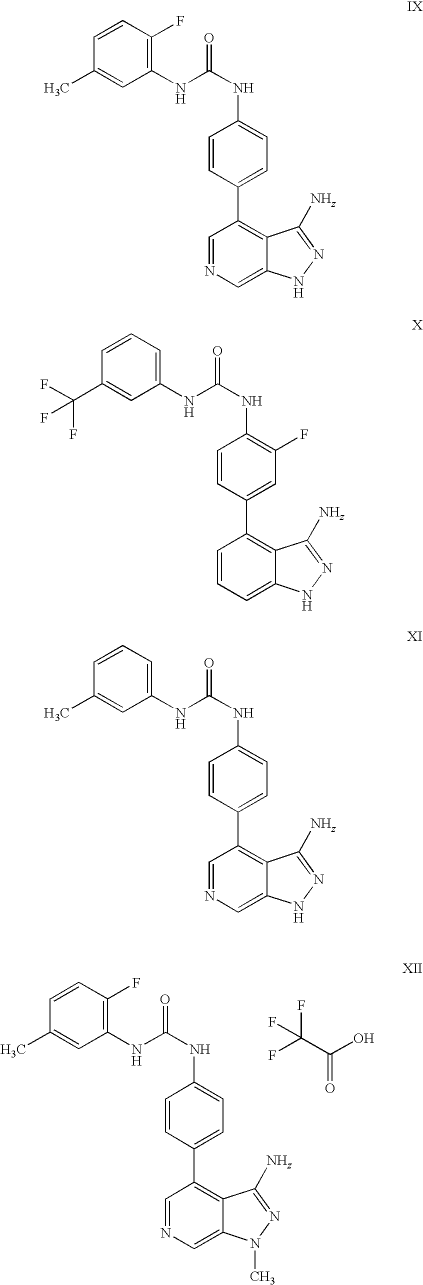

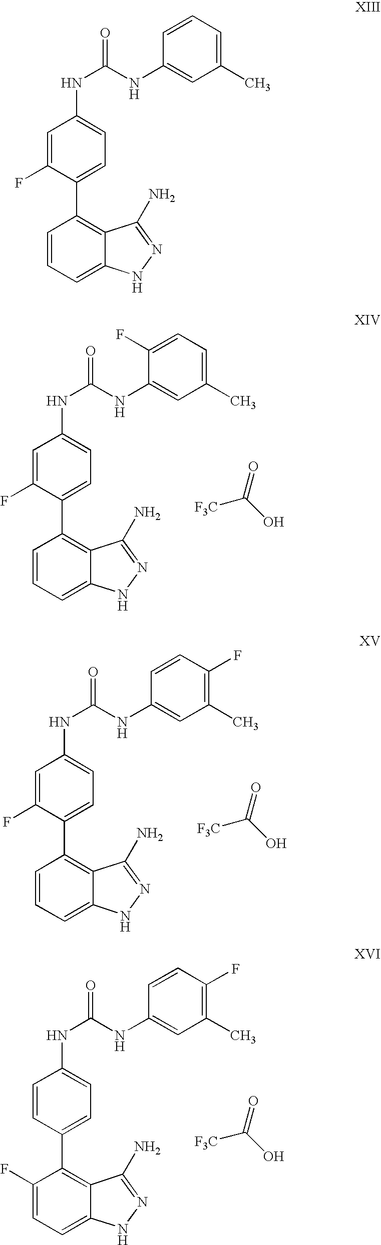

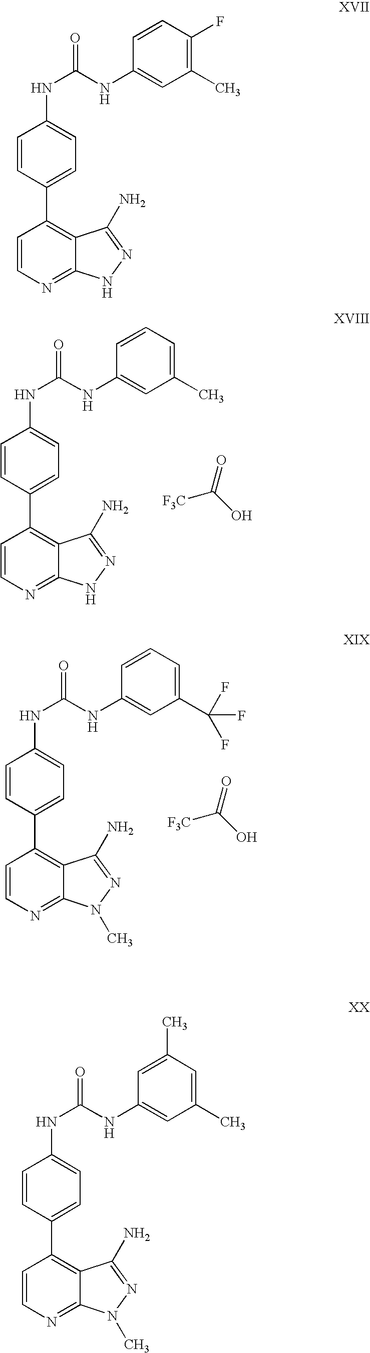

c) Antibody As used herein, the term “antibody” refers to an immunoglobulin molecule or immunologically active portion thereof, namely, an antigen-binding portion. Examples of immunologically active portions of immunoglobulin molecules include F(ab) and F(ab′)₂ fragments which can be generated, e.g., by treating an antibody with an enzyme such as pepsin. Examples of antibodies that can be used in the present disclosure include, but are not limited to, polyclonal antibodies, monoclonal antibodies, chimeric antibodies, human antibodies, humanized antibodies, recombinant antibodies, single-chain Fvs (“scFv”), affinity maturated antibodies, single chain antibodies, single domain antibodies, F(ab) fragments, F(ab′) fragments, disulfide-linked Fvs (“sdFv”), and antiidiotypic (“anti-Id”) antibodies, among others, and functionally active epitope-binding fragments of any of the above. The antibody may be of classes IgG, IgM, IgA, IgD or IgE, or fragments or derivatives thereof. For simplicity sake, an antibody against an analyte is frequently referred to as being either an “anti-analyte antibody”, or merely an “analyte antibody” (e.g., a human PlGF-1 antibody). The antibody may be derivatized by the attachment of one or more chemical, peptide, or polypeptide moieties known in the art. The antibody may be conjugated with a chemical moiety.

d) Human PlGF-1

The phrases “human PlGF-1” or “human PlGF-1 polypeptide” as used interchangeably herein refer to any full length (i.e., not a fragment thereof) human PlGF-1 sequence, either with or without a signal peptide. For example, the full length human PlGF-1 can be a 149 amino acid immature polypeptide with an 18 amino acid signal sequence having a centrally located PDGF-like domain with 8 conserved cysteine residues that form a cysteine knot structure. Alternatively, the human PlGF-1 can be a 131 amino acid mature polypeptide that does not contain the 18 amino acid signal sequence (such as described in the literature). The PlGF may exist in at least four alternatively spliced forms: PlGF-1, PlGF-2, PlGF-3 and PlGF-4. PlGF-2 and PlGF-4 may differ from other forms by the insertion of a heparin-binding domain in PlGF-2 and PlGF-4 that may result in increased association with the cell membrane or altered affinities for PlGF receptors. The amino acid sequence of PlGF is described in EP 0550519, the contents of which are herein incorporated by reference. Human PlGF polypeptide (e.g., polyamino acid) sequences are as found in nature, based on sequences found in nature, isolated, synthetic, semi-synthetic, recombinant, or other. In particular, PlGF as referred to herein is PlGF-1.

The disclosure herein encompasses a multitude of different human PlGF-1 polypeptide sequences as present and/or produced in a prokaryotic and/or eukaryotic background (e.g., with consequent optimization for codon recognition). In sum, the polypeptide and polynucleotide sequences may or may not possess or encode: (a) a signal peptide; and (b) other variations such as would be apparent to one skilled in the art.

e) Human PlGF-1 Fragment

As used herein, the term “human PlGF-1 fragment” refers to a polypeptide that comprises a part that is less than the entirety (i.e., not full length) of a human PlGF-1 (131 amino acids, referred to by some as the mature protein) or PlGF-1 including a signal peptide (149 amino acids, referred to by some as the immature protein). In particular, a human PlGF fragment comprises at least about 5 contiguous amino acids of human PlGF, at least about 10 contiguous amino acids residues of human PlGF; at least about 15 contiguous amino acids residues of amino acids of human PlGF; at least about 20 contiguous amino acids residues of human PlGF; at least about 25 contiguous amino acids residues of human PlGF, at least about 30 contiguous amino acid residues of amino acids of human PlGF, at least about 35 contiguous amino acid residues of human PlGF, at least about 40 contiguous amino acid residues of human PlGF, at least about 45 contiguous amino acid residues of human PlGF, at least about 50 contiguous amino acid residues of human PlGF, at least about 55 contiguous amino acid residues of human PlGF, at least about 60 contiguous amino acid residues of human PlGF, at least about 65 contiguous amino acid residues of human PlGF, at least about 70 contiguous amino acid residues of human PlGF, at least about 75 contiguous amino acid residues of human PlGF, at least about 80 contiguous amino acid residues of human PlGF, at least about 85 contiguous amino acid residues of human PlGF, at least about 90 contiguous amino acid residues of human PlGF, at least about 95 contiguous amino acid residues of human PlGF, at least about 100 contiguous amino acid residues of human PlGF, at least about 105 contiguous amino acid residues of human PlGF, at least about 110 contiguous amino acid residues of human PlGF, at least about 115 contiguous amino acid residues of human PlGF, at least about 120 contiguous amino acid residues of human PlGF, at least about 125 contiguous amino acid residues of human PlGF, at least about 130 contiguous amino acid residues of human PlGF, at least 135 contiguous amino acid residues of human PlGF, at least 140 contiguous amino acid residues of human PlGF or 144 contiguous amino acid residues of human PlGF.

An exemplary human PlGF-1 fragment comprises residues 21-149 of human PlGF-1, which is commercially available from R&D Systems, Inc., Minneapolis, Minn. (Catalog Number #P49763). This human PlGF-1 fragment can be used as a calibrator or control in an immunoassay for human PlGF-1 fragment or human PlGF-1 fragment.

f) Identical

“Identical” or “identity” as used herein in the context of two or more polypeptide or polynucleotide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation.

g) Monoclonal Antibody 264

As used herein, the phrase “monoclonal antibody 264” or “MAB264” refers to an unconjugated, mouse anti-human PlGF-1 monoclonal antibody from Clone 37203 which is commercially available from R&D Systems, Inc., Minneapolis, Minn. (Catalog Number MAB264).

h) Monoclonal Antibody 255

As used herein, the phrase “monoclonal antibody 255” or “MAB255” refers to an unconjugated, mouse anti-human PlGF-1 monoclonal antibody produced from murine hybridoma cell line 1-255-713 having American Type Culture Collection Accession No. PTA-8536, which is owned by Abbott Laboratories. Murine hybridoma cell line 1-255-713 was deposited with the American Type Culture Collection, 10801 University Blvd., Manassas, Va. 20110-2209 on Jul. 12, 2007 and assigned Accession No. PTA-8536. Monoclonal antibody 255 is described in U.S. Ser. No. 61/073,624 filed on Jun. 18, 2008 and U.S. Ser. No. 61/089,172 filed on Aug. 15, 2008, the contents of each of which are herein incorporated by reference.

The phrase “monoclonal antibody 255” or “MAB255” also refers to an unconjugated, mouse anti-human PlGF-1 monoclonal antibody that is produced by a subclone of murine hybridoma cell line 1-255-713, including monoclonal antibody produced by the murine hybridoma cell line 1-255-2675. Monoclonal antibody produced by murine hybridoma cell line 1-255-2675 is identical to the monoclonal antibody produced by murine hybridoma cell line 1-255-713. Murine hybridoma cell line 1-255-2675 is described in U.S. Ser. No. 12/485,114 filed on Jun. 16, 2009, the contents of which are herein incorporated by reference. When employed as conjugate (i.e., detection antibody), MAB255 can be used as a whole molecule or a fragment thereof (e.g., Fab′2 fragment).

i) Monoclonal Antibody 826

As used herein, the phrase “monoclonal antibody 826” or “MAB826” refers to an unconjugated, mouse anti-human PlGF-1 monoclonal antibody produced from murine hybridoma cell line 2-826-335 having American Type Culture Collection Accession No. PTA-8539, which is owned by Abbott Laboratories. Murine hybridoma cell line was deposited with the American Type Culture Collection, 10801 University Blvd., Manassas, Va. 20110-2209 on Jul. 12, 2007 and assigned Accession No. PTA-8536. Monoclonal antibody 826 is described in U.S. Ser. No. 61/073,624 filed on Jun. 18, 2008 and U.S. Ser. No. 61/089,172 filed on Aug. 15, 2008, the contents of each of which are herein incorporated by reference.

j) Pharmaceutical Composition

As used herein, the term “pharmaceutical composition” refers to any agent or drug, whether a small molecule (e.g., a drug containing an active agent, typically a non-peptidic) or biologic (e.g., a peptide or protein based drug, including any with modifications, such as, but not limited to pegylation) that can be used to treat a subject suffering from a disease or condition that requires treatment. Examples of pharmaceutical compositions, include, but are not limited to, ABT-869, analogs of ABT-869, hyperlipidemia drugs (including, but not limited to, niacin, fibrates (e.g., clofibrate, fenofibrate, fenofibric acid, simfrate, salts of fenofibric acid and any combinations thereof), ezetimibe, HMG-CoA reductase inhibitors (e.g., statins, such as, but not limited to rosuvastatin, simvastatin, and combinations thereof (including combinations with other hyperlipidemia drugs (e.g., simvastatin and ezetimibe)), anti-inflammatories, natriuretic peptide derivatives, etc. as well as any combinations thereof.

k) Polyclonal Antibody pB264

The phrases, “polyclonal antibody pB264”, “pB264”, “AF-264-PB”, “PAB264” or AB-264-PB as used interchangeably herein, refer to an unconjugated, goat anti-human PlGF-1 polyclonal antibody which is available from R&D Systems, Inc., Minneapolis, Minn. (Catalog Number AF-264-PB). Polyclonal antibody pB264 is produced in goats immunized with purified, E. coli-derived, recombinant human placenta growth factor. PlGF-1-specific IgG was purified by human PlGF affinity chromatography.

l) Predetermined Level

As used herein, the term “predetermined level” refers generally at an assay cutoff value that is used to assess diagnostic results by comparing the assay results against the predetermined level, and where the predetermined level already that has been linked or associated with various clinical parameters (e.g., monitoring whether a subject being treated with a drug has achieved an efficacious blood level of the drug, monitoring the response of a subject receiving treatment for cancer with an anti-cancer drug, monitoring the response of a tumor in a subject receiving treatment for said tumor, etc.). The predetermined level may be either an absolute value (such as in monoclonal antibody/polyclonal assays described in more detail herein) or a value normalized by subtracting the value obtained from a patient prior to the initiation of therapy (such as in monoclonal antibody/monoclonal antibodies as described in more detail herein). An example of a predetermined level that can be used is a baseline level obtained from one or more subjects that may optionally be suffering from one or more diseases or conditions. The present disclosure provides exemplary predetermined levels, and describes the initial linkage or association of such levels with clinical parameters for exemplary immunoassays as described herein. However, it is well known that cutoff values may vary dependent on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this description.

m) Substantially Identical

“Substantially identical,” as used herein may mean that a first and second sequence are at least from about 50% to about 99% identical over a region of from about 8 to about 100 or more residues (including, in particular, any range within from about 8 to about 100 residues).

n) Subject or Patient

As used herein, the terms “subject” and “patient” are used interchangeably. As used herein, the terms “subject” and “subjects” refer to an animal, in one aspect, a bird (for example, a duck or goose), in another aspect, a shark or whale, or in a further aspect, a mammal including, a non-primate (for example, a cow, pig, camel, llama, horse, goat, rabbit, sheep, hamsters, guinea pig, feline, canine, rat, and murine) and a primate (for example, a monkey, such as a cynomologous monkey, chimpanzee, and a human).

o) Test Sample

As used herein, the term “test sample” or “sample” generally refers to a biological material being tested for and/or suspected of containing an analyte of interest, such as a human PlGF-1 or human PlGF-1 fragment. The test sample may be derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

p) Toxicity Level

As used herein, the phrase “toxicity level” refers to a value in which a subject being treated with a pharmaceutical composition may begin to experience one or more adverse events or adverse side effects as a result of the treatment with said pharmaceutical composition.

q) Variant

“Variant” as used herein may mean a peptide or polypeptide that differs in amino acid sequence by the insertion, deletion, or conservative substitution of amino acids, but retain at least one biological activity. For purposes of this disclosure, “biological activity” includes the ability to be bound by a specific antibody. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol., 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated herein by reference. Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Variant may also refer to a protein that is (i) a portion of a referenced protein which may be from about 8 to about 100 or more amino acids (including, in particular, any range within from about 8 to about 100 residues); or (ii) a protein that is substantially identical to a referenced protein. A variant may also be a differentially processed protein, such as by proteolysis, phosphorylation, or other post-translational modification.

### B. PlGF-1 Assays and Methods of Treatment

The present disclosure also relates to assays for determining human PlGF-1 or human PlGF-1 fragment concentration in a test sample obtained from a subject. Assays contemplated include immunoassays (such as sandwich and competitive immunoassays), clinical chemistry assays and enzymatic assays. Preferably the human PlGF-1 or human PlGF-1 fragment measurement is done using an immunoassay, and more preferably, a sandwich immunoassay, which will be discussed in more detail herein.

Assays for determining human PlGF-1 or human PlGF-1 fragment concentration in a test sample obtained from a subject can comprise the steps of: (a) providing a test sample obtained from a subject; and (b) determining the concentration of human PlGF-1 or human PlGF-1 fragment in the test sample. A specific type of assay that can be performed for determining human PlGF-1 or human PlGF-1 fragment concentration is an immunoassay. Immunoassays can be conducted using any format known in the art, such as, but not limited to, a sandwich format, a competitive inhibition format (including both forward or reverse competitive inhibition assays) or in a fluorescence polarization format. As mentioned above, preferably, the immunoassay is in a sandwich format. Specifically, in one aspect of the present disclosure, at least two antibodies are employed to separate and quantify the human PlGF-1 or human PlGF-1 fragment in a test sample. More specifically, the at least two antibodies bind to certain epitopes of human PlGF-1 or human PlGF-1 fragment forming an immune complex which is referred to as a “sandwich”. Generally, in the immunoassays one or more antibodies can be used to capture the human PlGF-1 or human PlGF-1 fragment in the test sample (these antibodies are frequently referred to as a “capture” antibody or “capture” antibodies) and one or more antibodies can be used to bind a detectable (namely, quantifiable) label to the sandwich (these antibodies are frequently referred to as the “detection antibody”, “detection antibodies”, a “conjugate” or “conjugates”). In a sandwich assay, it is preferred that both antibodies binding to the human PlGF-1 or human PlGF-1 fragment are not diminished by the binding of any other antibody in the assay to its respective binding site. In other words, antibodies should be selected so that the one or more first antibodies brought into contact with a test sample or test sample extract suspected of containing human PlGF-1 or human PlGF-1 fragment do not bind to all or part of the binding site recognized by the second or subsequent antibodies, thereby interfering with the ability of the one or more second detection antibodies to bind to human PlGF-1 or human PlGF-1 fragment.

In one aspect of the present invention, excellent (namely, highly robust or high quality) immunoassays, particularly, sandwich assays, can be performed using a monoclonal antibody as a capture antibody and a polyclonal antibody as a detection antibody or a polyclonal antibody as a capture antibody and a monoclonal antibody as a detection antibody (referred to herein as “mono/poly assays”). An example of an exemplary immunoassay is one that employs as a capture antibody, monoclonal antibody 264, and, as the detection antibody, polyclonal antibody pB264. Optionally, a different commercially available antibody (e.g., other than monoclonal antibody 264) can be used as the first capture antibody and monoclonal antibody 264 can be used as a second or subsequent capture antibody. Alternatively, if monoclonal antibody 264 is being used as a first capture antibody, a different antibody (other than an monoclonal antibody 264, namely, other commercially available antibodies) can be used as a second capture antibody. Also optionally, a second detection antibody can be used in addition to polyclonal antibody pB264. Any commercially available antibody can be used as the second detection antibody. For example, polyclonal antibody pB264 can be used as a second detection antibody.

In another aspect of the present invention, excellent (namely, highly robust or high quality) immunoassays, particularly, sandwich assays, can be performed using a monoclonal antibody as both a capture antibody and as a detection antibody (referred to herein as “mono/mono assays”). An example of an exemplary immunoassay is one that employs as a capture antibody, monoclonal antibody 826 and, as the detection antibody, monoclonal antibody 255. Optionally, a different antibody, (e.g., other than monoclonal antibody 826) can be used as the first capture antibody and monoclonal antibody 826 can be used as a second or subsequent capture antibody. For example, monoclonal antibody 264 can be used as a first capture antibody and monoclonal antibody 826 can be used as the second or subsequent capture antibodies. Alternatively, if monoclonal antibody 826 is being used as a first capture antibody, a different antibody (other than an monoclonal antibody 826, namely, other commercially available antibodies) can be used as a second capture antibody. For example, monoclonal antibody 826 can be used as the first capture antibody and monoclonal antibody 264 can be used as the second capture antibody. Also optionally, a second detection antibody can be used in addition to monoclonal antibody 255. Any commercially available antibody can be used as the second detection antibody, including a polyclonal antibody, provided that the first detection antibody is a monoclonal antibody. For example, polyclonal antibody pB264 can be used as a second detection antibody, where a monoclonal antibody is used as a first detection antibody.

The sample being tested for (for example, suspected of containing) human PlGF-1 or human PlGF-1 fragment can be contacted with at least one capture antibody (or antibodies) and at least one detection antibody (which is either a second detection antibody or a third detection antibody) either simultaneously or sequentially and in any order. For example, the test sample can be first contacted with at least one capture antibody and then (sequentially) with at least one detection antibody. Alternatively, the test sample can be first contacted with at least one detection antibody and then (sequentially) with at least one capture antibody. In yet another alternative, the test sample can be contacted simultaneously with a capture antibody and a detection antibody.

In the sandwich assay format, a test sample suspected of containing human PlGF-1 or human PlGF-1 fragment is first brought into contact with an at least one first capture antibody under conditions which allow the formation of a first antibody-human PlGF-1 complex. If more than one capture antibody is used, a first multiple capture antibody-human PlGF-1 (or human PlGF-1 fragment) complex is formed. In a sandwich assay, the antibodies, preferably, the at least one capture antibody, are used in molar excess amounts of the maximum amount of human PlGF-1 expected in the test sample. For example, from about 5 μg/mL to about 1 mg/mL of antibody per mL of buffer (e.g., microparticle coating buffer) can be used.

Optionally, prior to contacting the test sample with the at least one capture antibody (for example, the first capture antibody), the at least one capture antibody can be bound to a solid support or solid phase which facilitates the separation the first antibody-human PlGF-1 complex from the test sample. Any solid support known in the art can be used, including but not limited to, solid supports made out of polymeric materials in the forms of wells of a reaction tray, test tubes or beads (for example, polystyrene beads, magnetic beads), nitrocellulose strips, membranes, microparticles (for example, latex particles, sheep and DURACYTES® (Abbott Laboratories, Abbott Park, Ill.; DURACYTES® are red blood cells that have been “fixed” by pyruvic aldehyde and formaldehyde)).

The solid phase also can comprise any suitable porous material with sufficient porosity to allow access by detection antibodies and a suitable surface affinity to bind antigens. Microporous structures generally are preferred, but materials with gel structure in the hydrated state may be used as well. Such useful solid supports include, but are not limited to, nitrocellulose and nylon. Such porous solid supports are preferably in the form of sheets of thickness from about 0.01 to 0.5 mm, preferably about 0.1 mm. The pore size may vary within wide limits, and preferably is from about 0.025 to about 15 microns, especially from about 0.15 to about 15 microns. The surface of such supports may be activated by chemical processes which cause covalent linkage of the antigen or antibody to the support. The irreversible binding of the antigen or antibody is obtained, however, in general, by adsorption on the porous material by poorly understood hydrophobic forces.

The antibody (or antibodies) can be bound to the solid support or solid phase by adsorption, by covalent bonding using a chemical coupling agent or by other means known in the art, provided that such binding does not interfere with the ability of the antibody to bind to human PlGF-1 or human PlGF-1 fragment. Alternatively, the antibody (or antibodies) can be bound with microparticles that have previously coated with streptavidin or biotin (for example, using Power-Bind™-SA-MP streptavidin coated microparticles, available from Seradyn, Indianapolis, Ind., with antibodies that have been biotinylated using means known in the art). Alternatively, the antibody (or antibodies) can be bound using microparticles that have been previously coated with anti-species specific monoclonal antibodies. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

After the test sample being tested for and/or suspected of containing human PlGF-1 or human PlGF-1 fragment is brought into contact with the at least one capture antibody (for example, the first capture antibody), the mixture is incubated in order to allow for the formation of a first antibody (or multiple antibody)-human PlGF-1 complex. The incubation can be carried out at a pH of from about 4.5 to about 10.0, at a temperature of from about 2° C. to about 45° C., and for a period from at least about one (1) minute to about eighteen (18) hours, preferably from about 1 to 20 minutes, most preferably from about 2-6 minutes. The immunoassay described herein can be conducted in one step (meaning the test sample, at least one capture antibody and at least one detection antibody are all added sequentially or simultaneously to a reaction vessel) or in more than one step, such as two steps, three steps, etc.

After formation of the (first or multiple) capture antibody-human PlGF-1 complex, the complex is then contacted with at least one detection antibody (under conditions which allow for the formation of a (first or multiple) capture antibody-human PlGF-1-(second or multiple) antibody detection complex). The at least one detection antibody can be the second, third, fourth, etc. antibodies used in the immunoassay. If the capture antibody-human PlGF-1 complex is contacted with more than one detection antibody, then a (first or multiple) capture antibody-human PlGF-1-(multiple) detection antibody complex is formed. As with the capture antibody (e.g., the first capture antibody), when the at least second (and subsequent) detection antibody is brought into contact with the capture antibody-human PlGF-1 complex, a period of incubation under conditions similar to those described above is required for the formation of the (first or multiple) capture antibody-human PlGF-1-(second or multiple) detection antibody complex. Preferably, at least one detection antibody contains a detectable label. The detectable label can be bound to the at least one detection antibody (e.g., the second detection antibody) prior to, simultaneously with or after the formation of the (first or multiple) capture antibody-human PlGF-1-(second or multiple) detection antibody complex. Any detectable label known in the art can be used. For example, the detectable label can be a radioactive label, such as, ³H, ¹²⁵I, ³⁵S, ¹⁴C, ³²P, ³³P, an enzymatic label, such as horseradish peroxidase, alkaline phosphatase, glucose 6-phosphate dehydrogenase, etc., a chemiluminescent label, such as, acridinium (e.g., acridium esters, acridinium SPSP (N10-(3-sulfopropyl)-N-(3-sulfopropyl, etc.), luminol, isoluminol, thioesters, sulfonamides, phenanthridinium esters, etc. a fluorescence label, such as, fluorescein (5-fluorescein, 6-carboxyfluorescein, 3′6-carboxyfluorescein, 5(6)-carboxyfluorescein, 6-hexachlorofluorescein, 6-tetrachlorofluorescein, fluorescein isothiocyanate, etc.), rhodamine, phycobiliproteins, R-phycoerythrin, quantum dots (zinc sulfide-capped cadmium selenide), a thermometric label or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997) and in Haugland, Handbook of Fluorescent Probes and Research Chemicals (1996), which is a combined handbook and catalogue published by Molecular Probes, Inc., Eugene, Oreg.

The detectable label can be bound to the antibodies either directly or through a coupling agent. An example of a coupling agent that can be used is EDAC (1-ethyl-3-(3-dimethylaminopropyl) carbodiimide, hydrochloride) that is commercially available from Sigma-Aldrich, St. Louis, Mo. Other coupling agents that can be used are known in the art. Methods for binding a detectable label to an antibody are known in the art. Additionally, many detectable labels can be purchased or synthesized that already contain end groups that facilitate the coupling of the detectable label to the antibody, such as, N10-(3-sulfopropyl)-N-(3-carboxypropyl)-acridinium-9-carboxamide, otherwise known as CPSP-Acridinium Ester or N10-(3-sulfopropyl)-N-(3-sulfopropyl)-acridinium-9-carboxamide, otherwise known as SPSP-Acridinium Ester.

The (first or multiple) capture antibody-human PlGF-1-(second or multiple) detection antibody complex can be, but does not have to be, separated from the remainder of the test sample prior to quantification of the label. For example, if the at least one capture antibody (e.g., the first capture antibody) is bound to a solid support or solid phase, such as, but not limited to, a well of a reaction tray, a bead or a microparticle, separation can be accomplished by removing the fluid (of the test sample) from contact with the solid support. Alternatively, if the at least first capture antibody is bound to a solid support it can be simultaneously contacted with the human PlGF-1 (or human PlGF-1 fragment)-containing sample and the at least one second detection antibody to form a first (multiple) antibody-human PlGF-1-second (multiple) antibody complex, followed by removal of the fluid (test sample) from contact with the solid support. If the at least one first capture antibody is not bound to a solid support, then the (first or multiple) capture antibody-human PlGF-1-(second or multiple) detection antibody complex does not have to be removed from the test sample for quantification of the amount of the label.

After formation of the labeled capture antibody-human PlGF-1-detection antibody complex (e.g., the first capture antibody-human PlGF-1-second detection antibody complex), the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified detecting the light emitted either visually or by using luminometers, x-ray film, high speed photographic film, a CCD camera, etc. Once the amount of the label in the complex has been quantified, the concentration of human PlGF-1 or human PlGF-1 fragment in the test sample is determined by use of a standard curve that has been generated using serial dilutions of human PlGF-1 or human PlGF-1 fragment of known concentration. Other than using serial dilutions of human PlGF-1 or human PlGF-1 fragment, the standard curve can be generated gravimetrically, by mass spectroscopy and by other techniques known in the art.

It goes without saying that the methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® Human PlGF-1 conjugate diluent (Abbott Laboratories, Abbott Park, Ill.) containing 2-(N-morpholino)ethanesulfonic acid (MES), another salt, protein blockers, an antimicrobial and detergent. An exemplary calibrator diluent is ARCHITECT® Human PlGF-1 calibrator diluent (Abbott Laboratories, Abbott Park, Ill.), which comprises a buffer containing MES, another salt, a protein blocker and an antimicrobial.

Furthermore, as previously mentioned, the methods and kits optionally are adapted for use on an automated or semi-automated system. Some of the differences between an automated or semi-automated system as compared to a non-automated system (e.g., ELISA) include the substrate to which the capture antibody is attached (which can impact sandwich formation and analyte reactivity), and the length and timing of the capture, detection and/or any optional wash steps. Whereas a non-automated format such as an ELISA may include a relatively longer incubation time with sample and capture reagent (e.g., about 2 hours) an automated or semi-automated format (e.g., ARCHITECT®) may have a relatively shorter incubation time (e.g., approximately 18 minutes for ARCHITECT®). Similarly, whereas a non-automated format such as an ELISA may incubate a detection antibody such as the conjugate reagent (Pb264) for a relatively longer incubation time (e.g., about 2 hours), an automated or semi-automated format (e.g., ARCHITECT®) may have a relatively shorter incubation time (e.g., approximately 4 minutes for the ARCHITECT®).

The assays of the present disclosure can be used to monitor whether a subject being administered a drug, such as ABT-869 or an analog of ABT-869, has obtained an efficacious (or optimal) blood level of said drug. In other words, the assays of the present disclosure allow the treating physician to determine whether or not the subject has received a sufficient amount of ABT-869 or an analog of ABT-869 to effectuate treatment. Such an assay involves contacting a first capture antibody that binds to human PlGF-1 or human PlGF-1 fragment with a test sample obtained from a subject receiving treatment with ABT-869 or an analog of ABT-869 to form a first capture antibody-human PlGF-1 complex. In mono/poly assays, the capture antibody optionally is monoclonal antibody 264. In mono/mono assays, the capture antibody optionally is monoclonal antibody 826. After the formation of the first capture antibody-human PlGF-1 complex, the test sample is then contacted with a second antibody that binds to human PlGF-1 or human PlGF-1 fragment and that has been conjugated to a detectable label to form a second capture antibody-human PlGF-1 detection complex. In mono/poly assays, the detection antibody optionally is polyclonal antibody pB264. In mono/mono assays, the detection antibody optionally is monoclonal antibody 225. The amount of the capture antibody-human PlGF-1 detection complex that has been formed is then determined by detecting the detectable label. The amount of capture antibody-human PlGF-1 detection complex present in the test sample correlates with the amount of human PlGF-1 or human PlGF-1 fragment in the test sample. The amount of human PlGF-1 or human PlGF-1 fragment in the test sample is then compared with a predetermined level. Specifically, if the concentration of human PlGF-1 or human PlGF-1 fragment determined in the test sample is lower than the predetermined level, then the subject is considered not to be receiving an efficacious (or optimal) amount of ABT-869 or analog of ABT-869. The treating physician may then make a decision to increase the amount of ABT-869 or analog of ABT-869 administered to the subject. However, if the concentration of human PlGF-1 or human PlGF-1 fragment determined in the test sample is the same as or higher than the predetermined level, then the subject is considered to be receiving an efficacious (or optimal) amount of ABT-869 or analog of ABT-869. For example, in mono/mono assays, a subject demonstrating an increase from the predetermined level (or baseline level (such as prior to treatment with ABT-869 or an analog of ABT-869)) of about 60 picograms per milliliter would be considered to be receiving an efficacious (or optimal) amount of ABT-869 or analog of ABT-869. More specifically, a subject demonstrating an increase from the predetermined level (such as a base line level (such as prior to treatment with ABT-869 or an analog of ABT-869)) in the range of about 60 picograms per milliliter to about 150 picograms per milliliter would be considered to be receiving an efficacious (or optimal) amount of ABT-869 or analog of ABT-869. An increase from the predetermined level (such as a baseline level) of 66.5 picograms per milliliter above the predetermined level (such as a baseline level) at about either 8 or 15 days after the subject first receives treatment with ABT-869 or an analog of ABT-869 at the steady state (namely at least about 3 days, at least about 5 days, at least about 7 days, at least about 10 days at least about 15 days, at least 20 days, at least 30 days, at least 35 days, at least 40 days, at least 45 days, at least 50 days, at least 60 days, etc.) after the subject first receives treatment with ABT-869 or an analog of ABT-869. The treating physician may then make the decision not to change or alter the amount of ABT-869 or analog of ABT-869 being administered to the subject; although, the treating physician could also decide to lower the amount of ABT-869 or analog of ABT-869 being administered to the subject as well for the reasons discussed further herein.

Additionally, in another aspect, in the mono/mono assays, if the toxicity level of the subject being treated with ABT-869 or an analog of ABT-869 reaches a range of 130 picograms per milliliter to 160 picograms per milliliter, the treating physician can reduce the amount of ABT-869 or analog of ABT-869 being administered to the subject. An exemplary toxicity level at which a treating physician can reduce the amount of ABT-869 or analog of ABT-869 being administered to the subject is 150 picograms per milliliter.

An exemplary predetermined level in mono/poly assays can be about 30 picograms per milliliter at about 24 hours after the subject first receives treatment with ABT-869 or an analog of ABT-869. Alternatively, in mono/poly assays, the predetermined level can be about 40 picograms per milliliter to about 75 picograms per milliliter at the steady state (namely at least about 3 days, at least about 5 days, at least about 7 days, at least about 10 days at least about 15 days, at least 20 days, at least 30 days, at least 35 days, at least 40 days, at least 45 days, at least 50 days, at least 60 days, etc.) after the subject first receives treatment with ABT-869 or an analog of ABT-869. An exemplary predetermined level in mono/mono assays can be about 66.5 picograms per milliliter at about 24 hours after the subject first receives treatment with ABT-869 or an analog of ABT-869.

The above described comparisons (also referred to as informational analysis) involving the amount of human PlGF-1 or human PlGF-1 fragment in the test sample and the predetermined level (such as a baseline level, can be made by an automated system, such as a software program or intelligence system that is part of, or compatible with, the automated or semi-automated equipment (e.g., computer platform) on which the above described assays are carried out. Alternatively, the above described comparisons can be done by a physician.

Subjects receiving treatment with ABT-869 or an analog of ABT-869 may be receiving treatment for cancer. The types of cancer include, but are not limited to, lung cancer, breast cancer, stomach cancer, bladder cancer, colon cancer, pancreatic cancer, ovarian cancer, prostate cancer, renal cancer, hepatocellular cancer, rectal cancer, hematopoietic malignancies, glioblastoma or infantile hemangioma. Alternatively, the subject may be suffering from autoimmune diseases, such as rheumatoid arthritis, thyroiditis, type 1 diabetes, multiple sclerosis, sarcoidosis, inflammatory bowel disease, Crohn's disease, myasthenia gravis and systemic lupus erythematosus; psoriasis, organ transplant rejection (e.g., kidney rejection, graft versus host disease), benign and neoplastic proliferative diseases or ocular diseases, such as, but not limited to, macular degeneration.

In another aspect, the assays of the present disclosure can be used to monitor the response of a subject receiving treatment for cancer with an anti-cancer drug. Such an assay involves contacting a first capture antibody that binds to human PlGF-1 or human PlGF-1 fragment with a test sample obtained from a subject suffering from cancer and receiving treatment with ABT-869 or an analog of ABT-869 to form a first capture antibody-human PlGF-1 complex. For mono/poly assays, the capture antibody is optionally monoclonal antibody 264. For mono/mono assays, the capture antibody optionally is monoclonal antibody 826. After the formation of the first capture antibody-human PlGF-1 complex, the test sample is then contacted with a second antibody that binds to human PlGF-1 or human PlGF-1 fragment and that has been conjugated to a detectable label to form a second capture antibody-human PlGF-1 detection complex. For mono/poly assays, the detection antibody optionally is polyclonal antibody pB264. For mono/mono assays, the detection antibody optionally is monoclonal antibody 255. The amount of the capture antibody-PlGF-1 detection complex that has been formed is then determined by detecting the detectable label. The amount of capture antibody-human PlGF-1 detection complex present in the test sample correlates with the amount of human PlGF-1 or human PlGF-1 fragment in the test sample. The amount of human PlGF-1 or human PlGF-1 test fragment in the test sample is then compared with a predetermined level. Specifically, if the concentration of human PlGF-1 or human PlGF-1 test fragment determined in the test sample is lower than the predetermined level, then the subject is considered not to be responding to treatment with ABT-869 or an analog of ABT-869 and the treatment with ABT-869 or an analog of ABT-869 is discontinued.

The treating physician may then make a decision to increase the amount of ABT-869 or analog of ABT-869 administered to the subject or to switch the subject to an entirely different anti-cancer drug to try and treat the cancer or may add another anti-cancer drug to the treatment regimen (e.g., administer to the subject ABT-869 or an analog of ABT-869 in combination with another anti-cancer drug). Additionally, the subject may then be administered a new (e.g., different) anti-cancer drug which is an anti-cancer drug other than ABT-869 or an analog of ABT-869. However, if the concentration of human PlGF-1 or human PlGF-1 fragment determined in the test sample is the same as or higher than the predetermined level, then the subject is considered to be responding to treatment with ABT-869 or analog of ABT-869. For example, in mono/mono assays, a subject demonstrating an increase from the predetermined level of about 60 picograms per milliliter would be considered to be responding to treatment with ABT-869 or analog of ABT-869. More specifically, a subject demonstrating an increase from the predetermined level of about 60 picograms per milliliter to about 150 picograms per milliliter would be considered to be responding to treatment with ABT-869 or analog of ABT-869. An increase of 66.5 picograms per milliliter above the predetermined level is particularly preferred. The treating physician may then make the decision not to change or alter the amount of ABT-869 or analog of ABT-869 being administered to the subject; although, the treating physician could also decide to lower the amount of ABT-869 or analog of ABT-869 being administered to the subject as well. For example, in mono/mono assays, a subject demonstrating an increase from the predetermined level (or baseline level) of about 60 picograms per milliliter would be considered to be responding to treatment with ABT-869 or analog of ABT-869. More specifically, a subject demonstrating an increase from the predetermined level (such as a base line level) in the range of about 60 picograms per milliliter to about 150 picograms per milliliter would be considered to be responding to treatment with ABT-869 or analog of ABT-869. An increase from the predetermined level (such as a baseline level) of 66.5 picograms per milliliter above the predetermined level (such as a baseline level) at about either 8 or 15 days after the subject first receives treatment with ABT-869 or an analog of ABT-869 at the steady state (namely at least about 3 days, at least about 5 days, at least about 7 days, at least about 10 days at least about 15 days, at least 20 days, at least 30 days, at least 35 days, at least 40 days, at least 45 days, at least 50 days, at least 60 days, etc.) after the subject first receives treatment with ABT-869 or an analog of ABT-869.

Additionally, also in the mono/mono assays, if the toxicity level of the subject being treated with ABT-869 or an analog of ABT-869 reaches a range of 130 picograms per milliliter to 160 picograms per milliliter, the treating physician can reduce the amount of ABT-869 or analog of ABT-869 being administered to the subject. An exemplary toxicity level at which a treating physician can reduce the amount of ABT-869 or analog of ABT-869 being administered to the subject is 150 picograms per milliliter.

An exemplary predetermined level in mono/poly assays can be about 30 picograms per milliliter at about 24 hours after the subject first receives treatment with ABT-869 or an analog of ABT-869. Alternatively, the predetermined level in mono/poly can be about 40 picograms per milliliter to about 75 picograms per milliliter at the steady state (namely at least about 3 days, at least about 5 days at least about 7 days, at least about 10 days, at least about 15 days, at least 20 days, at least 30 days, at least 35 days, at least 40 days, at least 45 days, at least 50 days, at least 60 days, etc.) after the subject first receives treatment with ABT-869 or an analog of ABT-869. An exemplary predetermined level in mono/mono assays can be about 66.5 picograms per milliliter at about 24 hours after the subject first receives treatment with ABT-869 or an analog of ABT-869. The above described comparisons (also referred to as informational analysis) involving the amount of human PlGF-1 or human PlGF-1 fragment in the test sample and the predetermined level (such as a baseline level, can be made by an automated system, such as a software program or intelligence system that is part of, or compatible with, the automated or semi-automated equipment (e.g., computer platform) on which the above described assays are carried out. Alternatively, the above described comparisons can be done by a physician. Subjects receiving treatment with ABT-869 or an analog of ABT-869 may be suffering from cancers such as lung cancer, breast cancer, stomach cancer, bladder cancer colon cancer, pancreatic cancer, ovarian cancer, prostate cancer, renal cancer, hepatocellular cancer, rectal cancer, hematopoietic malignancies, glioblastoma or infantile hemangioma.

In another aspect, the assays of the present disclosure can be used to determining whether a subject who is predisposed to a disease (e.g., a type of cancer) or who is suffering from a disease will respond to treatment with a drug. Such an assay involves contacting a first capture antibody that binds to human PlGF-1 or human PlGF-1 fragment with a test sample obtained from a subject who is predisposed or has a predisposition to a disease or that is suffering from at least one disease and receiving treatment with ABT-869 or an analog of ABT-869 to form a first capture antibody-human PlGF-1 complex. In mono/poly assays, the capture antibody optionally is monoclonal antibody 264. In mono/mono assays, the capture antibody optionally is monoclonal antibody 826. After the formation of the first capture antibody-human PlGF-1 complex, the test sample is then contacted with a second antibody that binds to human PlGF-1 or human PlGF-1 fragment and that has been conjugated to a detectable label to form a second capture antibody-human PlGF-1 detection complex. In mono/poly assays, the capture antibody optionally is polyclonal antibody pB264. In mono/mono assays, the detection antibody optionally is monoclonal antibody 255. The amount of the capture antibody-PlGF-1 detection complex that has been formed is then determined by detecting the detectable label. The amount of capture antibody-human PlGF-1 detection complex present in the test sample correlates with the amount of human PlGF-1 or human PlGF-1 fragment in the test sample. The amount of human PlGF-1 or human PlGF-1 test fragment in the test sample is then compared with a predetermined level. Specifically, if the concentration of human PlGF-1 or human PlGF-1 test fragment determined in the test sample is lower than the predetermined level, then a determination is made that the subject will not benefit from further or continued treatment with ABT-869 or an analog of ABT-869. The treating physician may then make a decision to see if the subject is eligible for further or continued treatment with a different drug. However, if the concentration of human PlGF-1 or human PlGF-1 fragment determined in the test sample is the same as or higher than the predetermined level, then a determination is made that the subject would or will benefit from further or continued treatment with ABT-869 or analog of ABT-869. Subjects that can be tested to determine whether or not they are eligible for treatment with a drug are subjects suffering from cancers such as lung cancer, breast cancer, stomach cancer, bladder cancer, colon cancer, pancreatic cancer, ovarian cancer, prostate cancer, renal cancer, hepatocellular cancer, rectal cancer, hematopoietic malignancies, glioblastoma or infantile hemangioma. Alternatively, the subject may be suffering from autoimmune diseases, such as rheumatoid arthritis, thyroiditis, type 1 diabetes, multiple sclerosis, sarcoidosis, inflammatory bowel disease, Crohn's disease, myasthenia gravis and systemic lupus erythematosus; psoriasis, organ transplant rejection (e.g., kidney rejection, graft versus host disease), benign and neoplastic proliferative diseases or ocular diseases, such as, but not limited to, macular degeneration.

The above assay can be used to monitor the progression of disease in subjects suffering from acute conditions. Acute conditions, also known as critical care conditions, refer to acute, life threatening diseases or other critical medical conditions involving the cardiovascular system (including, but not limited to, sepsis), central nervous stem and/or respiratory system. Typically, critical care conditions refer to those conditions requiring acute medical intervention in a hospital based setting (including, but not limited to, the emergency room, intensive care unit, trauma center or other emergent care setting) or administration by a paramedic or other field-based medical personnel. For critical care conditions, repeat monitoring is generally done within a shorter time frame, namely, minutes, hours or days (e.g., about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days or about 7 days), and the initial assay likewise is generally done within a shorter timeframe, e.g., about minutes, hours or days of the onset of the disease or condition.

The above assay can also be used to monitor the progression of disease in subjects suffering from chronic, or non-acute conditions. Non-critical care or, non-acute conditions, refers to conditions other than acute, life threatening disease or other critical medical conditions involving the cardiovascular system, central nervous system and/or respiratory system. Typically, non-acute conditions include those of longer-term or chronic duration, and include, e.g., ophthalmic conditions and cancer. For non-acute conditions, repeat monitoring generally is done with a longer timeframe, e.g., hours, days, weeks, months or years (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years), and the initial assay likewise generally is done within a longer time frame, e.g., about hours, days, months or years of the onset of the disease or condition.

In another aspect, the assays of the present invention can be used to monitor the response of a subject receiving treatment for cancer (e.g., the tumor) with a drug. Such an assay involves contacting a first capture antibody that binds to PlGF-1 with a test sample obtained from a subject that has one or more tumors and is receiving treatment with ABT-869 to form a first capture antibody-PlGF-1 complex. In mono/poly assays, the capture antibody optionally is monoclonal antibody 264. In mono/mono assays, the capture antibody is optionally monoclonal antibody 826. After the formation of the first capture antibody-PlGF complex, the test sample is then contacted with a second antibody that binds to PlGF-1 and that has been conjugated to a detectable label to form a second capture antibody-PlGF-1 detection complex. In mono/poly assays, the detection antibody is optionally polyclonal antibody pB264. In mono/mono antibodies, the detection antibody is optionally, monoclonal antibody 255. The amount of the capture antibody-PlGF-1 detection complex that has been formed is then determined by detecting the detectable label. The amount of capture antibody-PlGF-1 detection complex present in the test sample correlates with the amount of PlGF-1 in the test sample. The amount of PlGF-1 in the test sample is then compared with a predetermined level. Specifically, if the concentration of PlGF-1 determined in the test sample is lower than the predetermined level, then the patient is considered not to be responding to treatment with ABT-869. The treating physician may then make a decision to increase the amount of ABT-869 administered to the subject or to switch the subject to an entirely different drug to try and treat the cancer (e.g., tumor) or may add another drug to the treatment regiment (e.g., administer to the subject ABT-869 in combination with another drug). However, if the concentration of PlGF-1 determined in the test sample is the same as or higher than the predetermined level, then the patient is considered to be responding to treatment with ABT-869. The treating physician may then make the decision not to change or alter the amount of ABT-869 being administered to the subject; although, the treating physician could also decide to lower the amount of ABT-869 being administered to the subject as well. Subjects receiving treatment with ABT-869 may be suffering cancers (e.g., tumors) in connection with cancers such as lung cancer, breast cancer, stomach cancer, bladder cancer, colon cancer, pancreatic cancer, ovarian cancer, prostate cancer, renal cancer, hepatocellular cancer, rectal cancer, hematopoietic malignancies, glioblastoma or infantile hemangioma. Alternatively, the subject may be suffering from autoimmune diseases, such as rheumatoid arthritis, thyroiditis, type 1 diabetes, multiple sclerosis, sarcoidosis, inflammatory bowel disease, Crohn's disease, myasthenia gravis and systemic lupus erythematosus; psoriasis, organ transplant rejection (e.g., kidney rejection, graft versus host disease), benign and neoplastic proliferative diseases or ocular diseases, such as, but not limited to, macular degeneration.

In another aspect, the assays of the present disclosure can be used to confirm the biological activity of ABT-869 or an analog of ABT-869. Such an assay involves contacting a first capture antibody that binds to human PlGF-1 or human PlGF-1 fragment with a test sample obtained from a subject administered ABT-869 or an analog of ABT-869 to form a first capture antibody-human PlGF-1 complex. In mono/poly assays, the capture antibody is optionally monoclonal antibody 264. In mono/mono assays, the capture antibody optionally is monoclonal antibody 826. After the formation of the first capture antibody-human PlGF-1 complex, the test sample is then contacted with a second antibody that binds to human PlGF-1 or human PlGF-1 fragment and that has been conjugated to a detectable label to form a second capture antibody-human PlGF-1 detection complex. In mono/poly assays, the detection antibody is optionally polyclonal antibody pB264. In mono/mono assays, the detection antibody optionally is monoclonal antibody 255. The amount of the capture antibody-PlGF-1 detection complex that has been formed is then determined by detecting the detectable label. The amount of capture antibody-human PlGF-1 detection complex present in the test sample correlates with the amount of human PlGF-1 or human PlGF-1 fragment in the test sample. The amount of human PlGF-1 or human PlGF-1 test fragment in the test sample is then compared with a predetermined level (such as a baseline level prior to the subject being treated with ABT-869 or an analog of ABT-869). Specifically, if the concentration of human PlGF-1 or human PlGF-1 test fragment determined in the test sample is lower than the predetermined level, then a determination is made ABT-869 or analog of ABT-869 does not exhibit biological activity in the subject. However, if the concentration of human PlGF-1 or human PlGF-1 fragment determined in the test sample is the same as or higher than the predetermined level, then a determination is made that ABT-869 or analog of ABT-869 exhibits biological activity in the subject.

In still yet another aspect, the present disclosure relates to methods of treating subjects suffering from one or more types of cancer or one or more types of autoimmune diseases. For example, the cancer that can be treated according to the methods of the present invention can be one or more cancers selected from the group consisting of: lung cancer, breast cancer, stomach cancer, bladder cancer, colon cancer, pancreatic cancer, ovarian cancer, prostate cancer, renal cancer, hepatocellular cancer, rectal cancer, hematopoietic malignancies, glioblastoma and infantile hemangioma. Examples of autoimmune diseases that can be treated according to the methods of the present invention include, but are not limited to, rheumatoid arthritis, thyroiditis, type 1 diabetes, multiple sclerosis, sarcoidosis, inflammatory bowel disease, Crohn's disease, myasthenia gravis and systemic lupus erythematosus; psoriasis, organ transplant rejection (e.g., kidney rejection, graft versus host disease), benign and neoplastic proliferative diseases or ocular diseases, such as, but not limited to, macular degeneration.

The methods of treatment of the present disclosure involve obtaining a test sample from the subject suffering from at least one cancer or at least one autoimmune disease and who is receiving treatment with a predetermined amount of N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea. The predetermined amount of N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea being administered to the subject for whom the test sample is being obtained is the amount determined by the treating physician to be appropriate for the cancer or autoimmune disease being treated. For example, the predetermined amount of N-[4-(3-amino-1H-indazol-4-yl)phenyl]-N′-(2-fluoro-5-methylphenyl)urea or an analog of N-[4-(3-amino-1 H-indazol-4-yl)phenyl]-N′-(2-fluoro-5-methylphenyl)urea being administered or dosed to the subject can be 0.01 mg/kg, 0.05 mg/kg, 0.10 mg/kg, 0.25 mg/kg, 0.50 mg/kg, 0.75 mg/kg, 1.00 mg/kg, 1.25 mg/kg, 1.50 mg/kg, 1.75 mg/kg, 2.0 mg/kg, 2.25 mg/kg, 2.50 mg/kg, 2.75 mg/kg, 3.0 mg/kg, 3.25 mg/kg, 3.50 mg/kg, 3.75 mg/kg, 4.0 mg/kg, 4.25 mg/kg, 4.50 mg/kg, 4.75 mg/kg, 5.0 mg/kg, 5.25 mg/kg, 5.50 mg/kg, 5.75 mg/kg, 6.0 mg/kg, 6.25 mg/kg, 6.50 mg/kg, 6.75 mg/kg, 7.0 mg/kg, 7.25 mg/kg, 7.50 mg/kg, 7.75 mg/kg, 8.0 mg/kg, 8.25 mg/kg 8.50 mg/kg, 8.75 mg/kg, 9.0 mg/kg, 9.25 mg/kg, 9.50 mg/kg, 9.75 mg/kg, 10.00 mg/kg, etc.

Once the test sample has been obtained from the subject, the test sample is contacted with a first capture antibody that binds to PlGF-1 to form a first capture antibody-PlGF-1 complex. In mono/poly assays, the capture antibody optionally is monoclonal antibody 264. In mono/mono assays, the capture antibody is optionally monoclonal antibody 826. After the formation of the first capture antibody-PlGF complex, the test sample is then contacted with a second antibody that binds to PlGF-1 and that has been conjugated to a detectable label to form a second capture antibody-PlGF-1 detection complex. In mono/poly assays, the detection antibody is optionally polyclonal antibody pB264. In mono/mono antibodies, the detection antibody is optionally, monoclonal antibody 255. The amount of the capture antibody-PlGF-1 detection complex that has been formed is then determined by detecting the detectable label. The amount of capture antibody-PlGF-1 detection complex present in the test sample correlates with the amount of PlGF-1 in the test sample. The amount of PlGF-1 in the test sample is then compared with a predetermined level (such as a baseline level before the subject is treated with ABT-869 or an analog of ABT-869). Specifically, if the concentration of PlGF-1 determined in the test sample is lower than the predetermined level, then the patient is considered not to be responding to treatment with ABT-869. The treating physician may then make a decision to treat the subject by (a) increasing or adjusting the amount of ABT-869 or analog of ABT-869 being administered to the subject such that the amount of ABT-869 or analog of ABT-869 is higher then the predetermined amount of ABT-869 or analog of ABT-869 that has been previously been administered to the subject (For example, if the predetermined amount of ABT-869 or analog of ABT-869 being administered to the subject at the time the test sample is obtained is 0.05 mg/kg, the treating physician made decide to treat the subject by increasing the dosage of ABT-869 or analog of ABT-869 from 0.05 mg/kg to 0.25 mg/kg), (b) switching the subject to an entirely different drug to treat the cancer (e.g., tumor) or autoimmune disease; or (c) may add another drug to the treatment regimen (e.g., administer to the subject ABT-869 in combination with another drug). However, if the concentration of PlGF-1 determined in the test sample is the same as or higher than the predetermined level, then the patient is considered to be responding to treatment with ABT-869. The treating physician may then make the decision not to further treat the patient by changing or altering the amount of ABT-869 being administered to the subject; although, the treating physician can also decide to treat the patient by lowering the amount of ABT-869 being administered to the subject as well.

The above described comparisons (also referred to as informational analysis) involving the amount of human PlGF-1 or human PlGF-1 fragment in the test sample and the predetermined level (such as a baseline level, can be made by an automated system, such as a software program or intelligence system that is part of, or compatible with, the automated or semi-automated equipment (e.g., computer platform) on which the above described assays are carried out. Alternatively, the above described comparisons can be done by a physician.

Additionally, also in the mono/mono assays, if the toxicity level of the subject being treated with ABT-869 or an analog of ABT-869 reaches a range of 130 picograms per milliliter to 160 picograms per milliliter, the treating physician can reduce the amount of ABT-869 or analog of ABT-869 being administered to the subject. An exemplary toxicity level at which a treating physician can reduce the amount of ABT-869 or analog of ABT-869 being administered to the subject is 150 picograms per milliliter.

### C. Kits

The present disclosure also contemplates kits for detecting the presence of human PlGF-1 or human PlGF-1 fragment in a test sample and to be employed as a companion diagnostic for ABT-869 or an analog of ABT-869. Such kits can comprise one or more antibodies, including one or more of the antibodies described herein. More specifically, if the kit is a kit for performing an immunoassay, the kit optionally can contain (1) at least one capture antibody that specifically binds to human PlGF-1 or human PlGF-1 fragment; (2) at least one conjugate; and (3) one or more instructions for performing the immunoassay in connection with ABT-869 or an analog of ABT-869 (e.g., including for patient selection and/or dosage optimization). The antibodies described herein can be included in such a test kit as a capture antibody, as a detection antibody or both as a capture antibody and a detection antibody. For example, for use in mono/poly assays, monoclonal antibody 264 can be included in the kit as a capture antibody and polyclonal antibody pB264 can be included in the kit as a detection antibody. Alternatively, polyclonal antibody pB264 can be included in the kit as a capture antibody and monoclonal antibody 264 can be included in the kit as a detection antibody. In still yet another alternative, monoclonal antibody 264 or polyclonal antibody pB264 can be included in the kit as a capture antibody and a different antibody included in the kit as a detection antibody. In still yet another alternative, monoclonal antibody 264 or polyclonal antibody pB264 can be included in the kit as a detection antibody and a different antibody included in the kit as a capture antibody. Moreover, for example, for use in mono/mono assays, monoclonal antibody 826 can be included in the kit as capture antibody and monoclonal antibody 255 can be included in the kit as a detection antibody. Alternatively, monoclonal antibody 255 can be included in the kit as a capture antibody and monoclonal antibody 826 can be included in the kit as a detection antibody. In still yet another alternative, monoclonal antibody 826 or monoclonal antibody 255 can be included in the kit as a capture antibody and a different antibody included in the kit as a detection antibody. In still yet another alternative, monoclonal antibody 826 or monoclonal antibody 255 can be included in the kit as a detection antibody and a different antibody included in the kit as a capture antibody.

Optionally, the kit can also contain at least one calibrator or control. Any calibrator or control can be included in the kit. Preferably, however, the calibrator or control is a human PlGF-1 fragment. More preferably, the calibrator or control comprises an isoform of human PlGF-1 that comprises residues 21-149 of human PlGF-1 and is available from R&D Systems, Inc., Minneapolis, Minn. (Catalog Number #P49763). Optionally, the kit can also contain at least one sample collection tube.

Thus, the present disclosure further provides for diagnostic and quality control kits comprising one or more antibodies described herein. Optionally the assays, kits and kit components of the invention are optimized for use on commercial platforms (e.g., immunoassays on the Prism®, AxSYM®, ARCHITECT® and EIA (Bead) platforms of Abbott Laboratories, Abbott Park, Ill., as well as other commercial and/or in vitro diagnostic assays). Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories, Abbott Park, Ill.) electrochemical immunoassay system that performs sandwich immunoassays for several cardiac markers, including TnI, CKMB and BNP. Immunosensors and methods of operating them in single-use test devices are described, for example, in U.S. Patent Applications 20030170881, 20040018577, 20050054078 and 20060160164 which are incorporated herein by reference. Additional background on the manufacture of electrochemical and other types of immunosensors is found in U.S. Pat. No. 5,063,081 which is also incorporated by reference for its teachings regarding same.

Optionally the kits include quality control reagents (for example, sensitivity panels, calibrators, and positive controls). Preparation of quality control reagents is well known in the art, and is described, e.g., on a variety of immunodiagnostic product insert sheets.

In another embodiment, the present disclosure provides for a quality control kit comprising one or more antibodies described herein for use as a sensitivity panel to evaluate assay performance characteristics and/or to quantitate and monitor the integrity of the antigen(s) used in the assay.

The kits can optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), may also be included in the kit. The kit may additionally include one or more other controls. One or more of the components of the kit may be lyophilized and the kit may further comprise reagents suitable for the reconstitution of the lyophilized components.

The various components of the kit optionally are provided in suitable containers. As indicated above, one or more of the containers may be a microtiter plate. The kit further can include containers for holding or storing a sample (e.g., a container or cartridge for a blood or urine sample). Where appropriate, the kit may also optionally contain reaction vessels, mixing vessels and other components that facilitate the preparation of reagents or the test sample. The kit may also include one or more instruments for assisting with obtaining a test sample, such as a syringe, pipette, forceps, measured spoon, or the like.

The kit further can optionally include instructions for use, which may be provided in paper form or in computer-readable form, such as a disc, CD, DVD or the like.

Exemplary kits according to the present invention can be used to determine whether a subject receiving treatment with a drug has obtained an efficacious blood level of that drug, to monitor response of a subject receiving treatment for cancer with an anti-cancer drug, to determine whether a subject who is predisposed to disease or who is suffering from a disease will respond to treatment with a drug, to monitor progression of disease in a subject being treated with a drug, to confirm biological activity of a drug like ABT-869 or analog of ABT-869 in a subject or as part of a treatment regimen to treat a subject suffering from cancer or an autoimmune disease.

### D. Adaptations of the Methods of the Present Disclosure

The invention as described herein also can be adapted for use in a variety of automated and semi-automated systems (including those wherein the solid phase comprises a microparticle), as described, e.g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM®, IMX, PRISM®, and Quantum II instruments, as well as other platforms. Moreover, the invention optionally is adaptable for the Abbott Laboratories commercial Point of Care (i-STAT™) electrochemical immunoassay system for performing sandwich immunoassays. Immunosensors, and their methods of manufacture and operation in single-use test devices are described, for example in, U.S. Pat. No. 5,063,081, U.S. Patent Application 2003/0170881, U.S. Patent Application 2004/0018577, U.S. Patent Application 2005/0054078, and U.S. Patent Application 2006/0160164, which are incorporated in their entireties by reference for their teachings regarding same.

By way of example and not of limitation, examples of the present disclosure shall now be given.

### Example 1

### Study Involving Patients with Refractory Solid Malignancies Receiving Treatment with ABT-869 Including Human PlGF-1 Analysis

### A. Patients and Methods

Eligible patients were 18 years or older with histologically confirmed advanced non-haematologic malignancy refractory to or with no standard therapy. Other criteria included having ECOG (Eastern Cooperative Oncology Group) Performance Score of 0-2, measurable disease by CT or MRI, and laboratory values fulfilling the following criteria: haemoglobin ≧9.0 g/dL, platelet ≧100,000/μL, absolute neutrophil count (ANC) ≧1,000/μL, creatinine ≦1.5× upper normal limit (ULN), bilirubin, AST and ALT ≦1.5×ULN of institution's normal range.

The main exclusion criteria were: anticancer therapy within the previous 28 days; life expectancy of less than 12 weeks; history of central nervous system metastases; significant proteinuria; uncontrolled hypertension; left ventricular ejection fraction <50%; active signs of bleeding; or anticoagulation therapy for therapeutic intent. The study received approval by the institutional ethics review board and all patients provided written informed consent.

### B. Study Design and Drug Treatment

This study was designed as a single-arm, open-label Phase I trial and was conducted in three segments (A, B and C). Segment A was a sequential dose-escalation study with primary intent to define the maximum tolerable dose (MTD), segment B involved expansion of the next lower dose to a total of 12 patients to further evaluate tolerability, and segment C was to study the tolerability and pharmacodynamics of a lower dose cohort to better define dose-effect relationships. In all three segments, patients received ABT-869 until tumor progression or occurrence of dose-limiting toxicity (DLT).

The starting dose of 10 mg was obtained by applying a safety factor of 5 to the no-observed-adverse-event-level dosage used in the one-month rat study, which was the more sensitive species. A dose of 10 mg was selected as the projected Cₘₐₓ and AUC (0.05 μg/ml and 0.75 μg·hr/ml, respectively) had a safety margin for observed toxicity of at least 4.2 for a 70 kg person based on a body surface area scaling. ABT-869 was self-administered as a continuous daily oral dosage at night (except on days 1 and 15 when drug was administered in the morning for assessment of PK) in treatment periods of 21 days. No drug was administered on day 14. As ABT-869 lacks high aqueous solubility, the study drug was diluted in 60 mLs of Ensure Plus®. Preliminary PK at doses if 10 mg showed a modest correlation between oral clearance and body-weight; thus subsequent dose escalations in segment A were based on body-weight.

Dose escalation was planned in cohorts of three patients each, and cohort expansion to six patients was planned if DLT occurred in 1 of 3 patients during the first treatment period. DLT was defined as follows: grade 3 fatigue; grade 3 proteinuria; persistent grade 3 hypertension despite intervention; grade 3 neutropenia with fever; grade 4 neutropenia >7 days; grade 4 thrombocytopenia; any other related grade 3 or 4 adverse events; and any unexpected grade 2 toxicity of possible or probable relationship to treatment, which required dose modification or delay of more than one week. Dose escalation was stopped if 2 or more patients out of 6 in a dose cohort experienced DLT within the first treatment period; that dose would be considered the MTD.

### C. Platelet-Free Plasma for Human PlGF-1 Analysis

Approximately 4 mL of blood was collected by venipuncture into a 4 mL EDTA (purple cap) on TP1D1 pre-dose and at 6 and 24 hour post-dose and TP1D15 pre-dose and at 6 hour post-dose. Additional specimens were collected on TP2D1 (Day 22) and TP3D1 (Day 43) and at the end of TP 4, 6 and 8. A sample was collected at the time of the Final Visit. A sample was also collected at the time of a Grade 3 toxicity of asthenia. The collection tube was inverted (2-3 times) to reduce the likelihood of clot formation. The tube was centrifuged at 1500×g for 15 min. at 2-8° C. within 30 minutes of collection. The plasma was split into two 1.5 mL aliquots and centrifuged at 10,000×g for 10 minutes at 2-8° C. to complete platelet removal. The plasma was immediately transferred (supernatant) to two appropriately labeled 2 mL cryovials and frozen at −70° C. The samples were stored at −70° C. until notification was received to ship the batches. The complete process of centrifugation, transfer to cryovial and freezing was accomplished in less than 1 hour from blood draw.

### D. Tumor Response Evaluation and Safety

Baseline CT imaging (CT) was performed within 4 weeks before the ABT-869 treatment, and repeated every two treatment periods (6 weeks). Lesions were evaluated using RECIST (Response Evaluation Criteria in Solid Tumors). Adverse events were recorded and graded according to CTC (Common Terminology Criteria) version 3.0. Physical examination, complete blood counts, serum chemistries including troponin T, urinalysis and serial electrocardiograms were assessed at least weekly for the first 4 treatment periods. ACTH tests and Multiple Gated Acquisition Scan scans were done at baseline and after every four treatment periods to assess adrenal and cardiac safety with repeated dosing.

### E. Pharmacokinetic Assessment

PK sampling was performed on days 1, 15 at the following time points: pre-dose, 0.25, 0.5, 1, 2, 3, 4, 6, 8 and 24 hours post-dosing. ABT-869 and its metabolite concentrations in plasma were determined using a validated method based on triple quadrupole tandem mass spectrometry with a lower limit of quantification of 1.0 ng/mL. Pharmacokinetic parameters and metabolites were analyzed with non-compartmental methods based on WINNONLIN (Pharsight Corp., Cary, N.C.). The area under the concentration-time curve (AUC) was estimated using the log-linear trapezoidal option and by extrapolation of the terminal curve to infinity including at least the last 3 concentration points; oral clearance, half-life (t½) and volume of distribution at steady state (VDss) were calculated.

### F. Pharmacodynamic Assessments

Samples for biomarkers of angiogenesis were collected on days 1 (baseline and 6, 24 hours post dosing), 15, 21 and 42 and at the end of the 4^{th} and 6^{th} treatment periods. Plasma VEGF was measured using commercial ELISA kits from R& D Systems (Minneapolis, Minn.). A prototype, automated immunoassay designed to measure PlGF-1 on the Abbott ARCHITECT® instrument system (Abbott Laboratories, Abbott Park, Ill.) was used in this study. The immunoassay is configured in a two-step sandwich assay format using monoclonal antibody 264 as the capture antibody and polyclonal antibody pB264 as the detection antibody, and using recombinant human PlGF-1 (R&D Systems, Inc., Minneapolis, Minn., Catalog Number 264-PG/CF) as calibrator. The circulating (or free) PlGF-1 present in blood specimens was captured using anti-PlGF-1 monoclonal antibody 264 coated paramagnetic microparticles (which were made using routine techniques known in the art) with detection via affinity purified anti-PlGF-1 polyclonal antibodies pB264 that were labeled with acridinium. The dynamic range of the assay was 0-1500 pg/mL.

Generally, paramagnetic latex microparticles (4.7 μM), derivatized with carboxyl functional groups, were coated with anti-PlGF-1 monoclonal antibody at an antibody concentration demonstrated to be sufficient to maximize the level of antibody absorbed to the surface area of the microparticles (2% solids by weight) in 50 mM 2-(N-morpholino)ethanesulfonic acid (MES), pH 5.5. After 10 minutes, the non-absorbed antibody was removed by washing the particles multiple times with MES buffer. Following washing of the particles, EDAC (1-ethyl-3-(3-dimethylaminopropyl) carbodiimide, hydrochloride, Sigma-Aldrich, St. Louis, Mo.) was added and allowed to react, forming a covalent coupling of the antibody molecules to the particles. The particles were then washed with phosphate buffered saline to stop the reaction and remove unreacted EDAC. The particles were then diluted to 0.1% in buffer for use in an automated immunochemical analyzer.

Acridinium-labeled anti-PlGF-1 antibody was prepared by incubating a polyclonal antibody (alternatively a monoclonal antibody could be used) with an acridinium designated SPSP (N10-(3-sulfopropyl)-N-(3-sulfopropyl) acridinium-9-carboxamide at a molar ratio of acridinium to antibody ranging from 1 to 100. Unconjugated acridinium was then separated from the acridinium-labeled antibody conjugate by size chromatography. The purified conjugate was then diluted in buffer to a concentration yielding the maximum signal to noise ratio in the assay.

Circulating populations of activated, apoptotic and progenitor endothelial cells were measured using a modified flow cytometry-based method previously described in Mancuso P, et al:, Blood 97:3658-3661 (2001) and Yee, K. W., et al., Clin Cancer Res., 11: 6615-6624 (2005).

### G. Statistical Analyses

For PlGF-1 analysis, the statistical analysis was performed using the JMP 7.0.1 statistical analysis program produced by the SAS Institute. Univariate Survival Analysis with log rank analysis was performed to identify an optimal PlGF-1 threshold which stratified patients for time on therapy (indicative of therapeutic benefit). Groups are shown for the Day 15 time point as either all patients, >40 pg/mL or <40 pg/mL, with clear differences in the time to progression for each group.

Patients were classified as having poor effect if tumor progression occurred at or before treatment period 4 and good effect if progression had not occurred at that time.

### H Results

### Patient Characteristics

Thirty-three patients were recruited into the study (See, Table 1, below).

TABLE 1

| Patient Characteristic | |
|---|---|
| Sex (Males/Females) | 16/17 |
| Age Median (range) years | 56 (29-76) |
| Eastern Cooperative Group Performance Status | |
| 0 | 18 |
| 1 | 13 |
| 2 | 2 |
| Tumour Sites | |
| Non-small cell lung | 8 |
| Colorectal | 7 |
| Hepatocellular | 4 |
| Ovary | 3 |
| Breast | 2 |
| Neuroendocrine | 2 |
| Endometrial Sarcoma | 2 |
| Others* | 5 |
| Prior lines of treatment | |
| 0-2 | 13 |
| >2 | 20 |

*Other tumor types included one patient each with urachal carcinoma, soft tissue sarcoma, renal cell carcinoma, nasopharyngeal carcinoma and primitive neuroectodermal tumor.

Treatment was discontinued due to disease progression and adverse events in 18 patients, of which 13 subjects had radiographic disease progression; 5 patients discontinued due to adverse events related to ABT-869; 1 patient discontinued due to an adverse event unrelated to ABT-869; 1 patient discontinued due to an adverse event with an unknown relationship to ABT-869; 4 patients continued to receive treatment with clinical benefit at the time of writing. Median overall treatment duration (excluding days without taking ABT-869) for all dose cohorts was 84 days (range 4-694 days). There were no treatment-related deaths.

### I. Toxicity and Tolerability of Repeated Dosing

The most common drug-related adverse events were fatigue, proteinuria, hypertension, myalgia, skin toxicity (hand and foot blisters) and oral hypersensitivity, and these toxicities increased in frequency and intensity with increasing doses (See, Tables 2A and 2B and 3, below).

TABLE 2A

| | (N = 33) | | | |
|---|---|---|---|---|
| | Any Grade | | Grade 3 | |
| | No. of | | No. of | |
| Adverse Event | Patients | % | Patients | % |
| Patients reporting adverse events | 33 | 100 | 16 | 48 |
| Fatigue | 28 | 85 | 4 | 12 |
| Proteinuria | 26 | 79 | 5 | 15 |
| Skin (Hand & Foot) | 20 | 61 | 1 | 3 |
| Hypertension | 18 | 55 | 4 | 12 |
| Myalgia | 16 | 48 | 0 | 0 |
| Mouth dryness/hypersensitivity | 14 | 42 | 0 | 0 |
| Diarrhoea | 11 | 33 | 1 | 3 |
| Anorexia | 11 | 33 | 0 | 0 |
| Nausea/Vomiting | 10 | 30 | 0 | 0 |
| Skin Rash | 9 | 27 | 0 | 0 |
| Haemoptysis | 5 | 15 | 0 | 0 |
| Abdominal pain | 3 | 9 | 1 | 3 |
| Pneumothorax | 3 | 9 | 0 | 0 |
| Back pain | 3 | 9 | 0 | 0 |

TABLE 2B

| | | ABI 869 Dose | | | |
|---|---|---|---|---|---|
| | | | | 0.25 | 0.3 |
| | CTC | 0.1 mg/kg | 10 mg | mg/kg | mg/kg |
| Toxicity | Grade | (N = 12) | (N = 6) | (N = 12) | (N = 3) |
| Fatigue | 3 | 0 | 1* | 0 | 0 |
| Proteinuria | 3 | 2^{†}* | 0 | 2* | 1* |
| Hypertension | 2 | 0 | 0 | 2* | 0 |
| | 3 | 1 | 0 | 1 | 2^{#} |
| Pneumothorax | 2 | 0 | 0 | 2 | 0 |
| Gastroenteritis | 3 | 0 | 0 | 1 | 0 |
| Abdominal Pain | 3 | 0 | 0 | 1 | 0 |
| Palmar-plantar | 2 | 0 | 0 | 0 | 1 |
| erythrodysesthesia | 3 | 0 | 0 | 1 | 0 |
| (PPE) | | | | | |

*DLTs during the first treatment period.

TABLE 3

| Pharmacokinetic | | Dose | | | |
|---|---|---|---|---|---|
| Parameter (Units) | | 0.10 mg/kg | 10 mg | 0.25 mg/kg | 0.3 mg/kg |
| | | Study Day 1 | | | |
| N | | 11 | 6 | 12 | 3 |
| Tₘₐₓ | (h) | 3.5 ± 1.5 | 3.3 ± 1.5 | 2.7 ± 0.8 | 2.0 ± 0.0 |
| Cₘₐₓ | (μg/mL) | 0.12 ± 0.05 | 0.21 ± 0.12 | 0.25 ± 0.09 | 0.34 ± 0.09 |
| Cₘₐₓ/D | (μg/mL/mg) | 0.020 ± 0.007 | 0.021 ± 0.012 | 0.019 ± 0.006 | 0.020 ± 0.008 |
| AUC_{∞} | (μg · h/mL) | 3.1 ± 1.4 | 4.1 ± 2.2 | 5.8 ± 2.9 | 7.9 ± 2.0 |
| AUC_{∞}/D | (μg · h/mL/mg) | 0.51 ± 0.21 | 0.41 ± 0.22 | 0.41 ± 0.19 | 0.47 ± 0.19 |
| t_{1/2} | (h) | 19.0 ± 5.6 | 14.4 ± 4.6 | 18.9 ± 6.2 | 22.0 ± 2.4 |
| CL/F | (L/h) | 2.3 ± 0.9 | 3.0 ± 1.4 | 3.0 ± 1.3 | 2.4 ± 0.8 |
| | | Study Day 15 | | | |
| N | | 11 | 6 | 11 | 3 |
| Tₘₐₓ | (h) | 3.7 ± 1.5 | 3.0 ± 0.0 | 3.5 ± 1.0 | 3.3 ± 0.6 |
| Cₘₐₓ | (μg/mL) | 0.14 ± 0.05 | 0.22 ± 0.17 | 0.31 ± 0.12 | 0.39 ± 0.17 |
| Cₘₐₓ/D | (μg/mL/mg) | 0.024 ± 0.008 | 0.026 ± 0.019 | 0.022 ± 0.006 | 0.022 ± 0.008 |
| AUC₂₄ | (μg · h/mL) | 2.1 ± 0.9 | 3.0 ± 1.5 | 4.3 ± 2.1 | 5.3 ± 1.5 |
| AUC₂₄/D | (μg · h/mL/mg) | 0.35 ± 0.15 | 0.35 ± 0.20 | 0.30 ± 0.08 | 0.30 ± 0.07 |

Pharmacokinetics at day 1 and day 15 of ABT-869 treatment.
Abbreviations:
Tmax- time to maximum concentration;
Cmax- Maximum concentration;
D-actual dose;
AUC∞-area under the concentration-time curve extrapolated to infinity;
t½- half-life;
CL/F- oral clearance.

The most common drug-related adverse events were fatigue, proteinuria, hypertension, myalgia, skin toxicity (hand and foot blisters) and oral hypersensitivity, and these toxicities increased in frequency and intensity with increasing doses (Tables 2B). DLTs in the first treatment period (3 weeks) in segment A were grade 3 fatigue in 1 of 6 patients at 10 mg/day, none at 0.25 mg/kg/day, grade 3 hypertension in 1 patient and grade 3 proteinuria in another patient at the MTD of 0.3 mg/kg/day. In segment B, the cohort of 0.25 mg/kg/day was expanded to a total of 12 patients, with a first treatment period DLT of grade 3 proteinuria observed in a patient with a diagnosis of quiescent systemic lupus erythematosis, and one patient with grade 3 hypertension. Repeated dosing at 0.25 mg/kg/day after the first treatment period resulted in dose reductions for drug-related toxicity in 7 patients, including one patient with both grade 3 proteinuria and grade 2 hypertension (Cycle 3), one patient with grade 3 foot blisters (Cycle 2), one patient with symptomatic grade 2 hypertension (Cycle 2), one patient with grade 3 gastroenteritis (Cycle 5) and one with grade 3 abdominal pain (Cycle 3). In addition two patients (one each in Cycle 1 and Cycle 3) experienced grade 2 or grade 3 pneumothorax attributed to therapy induced cavitation of lung nodules.

Since the starting dose of 10 mg achieved or exceeded the minimum PK targeted for efficacy and antitumor effects were observed, a lower dose of 0.1 mg/kg/day dose was studied in an additional 12 patients in segment C. Compared to 0.25 mg/kg/day, the 0.1 mg/kg/day dose had fewer episodes of grade 2 or higher toxicities, with DLT observed in 1 patient who experienced grade 3 proteinuria in the first treatment period and a patient each with grade 3 hypertension and proteinuria after treatment period 1. The incidence of hypertension was dose-dependent, with an incidence of hypertension of at least grade 2 of 25% at 0.1 mg/kg/day compared to 50% at 0.25 mg/kg/day. In addition, the change in mean blood pressure correlated with dose (data not shown). Hypertension responded to standard antihypertensive therapy with angiotensin converting enzyme inhibitors, β blockers and calcium channel blockers and no patient developed hypertensive crisis. Skin blisters and proteinuria resolved after reduction or discontinuation of ABT-869. No significant hematological or cardiac toxicities were observed in any of the patients treated. Overall, 14 patients (42%) required dose reductions due to adverse events. Four patients have been on the study for ≧12 months without major cumulative toxicities after dose stabilization.

### J. Pharmacokinetic Evaluation

Pharmacokinetic data was available for 32 and 31 patients on days 1 and 15, respectively. Doses above 0.1 mg/kg achieved plasma exposures (>2.7 μg h/mL) that were relevant for antitumor activity based on a preclinical murine HT1080 fibrosarcoma model. (See, Albert, et al., Molec. Cancer Ther. 5(4), 996-1006 (2006). Over the studied dose range, absorption and elimination of ABT-869 were linear (See, Table 3) and pharmacokinetics of ABT-869 were dose-proportional and time-invariant. The mean time to Cₘₐₓ, t_{1/2} and oral clearance was 2.9 h (range 2-8 h), 18.6±5.7 h and 2.7±1.2 L/h, respectively. Day 15 accumulation ratio was 1.1±0.4. The main systemic metabolite was the carboxylate metabolite. From the urinary recovery analysis of 4 patients, <10% of ABT-869 dose was recovered in the urine as unchanged drug and carboxylate metabolite. At final analysis, CL/F did not correlate with body weight. No correlation with BSA, creatinine clearance, or baseline AST/ALT/Albumin was observed.

### K. Efficacy

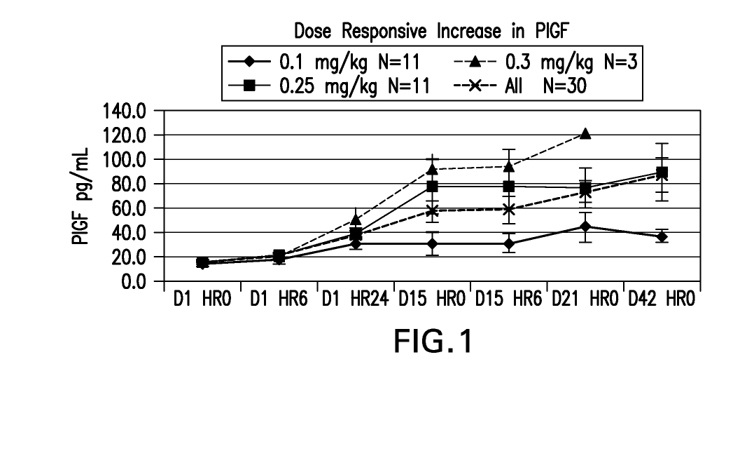

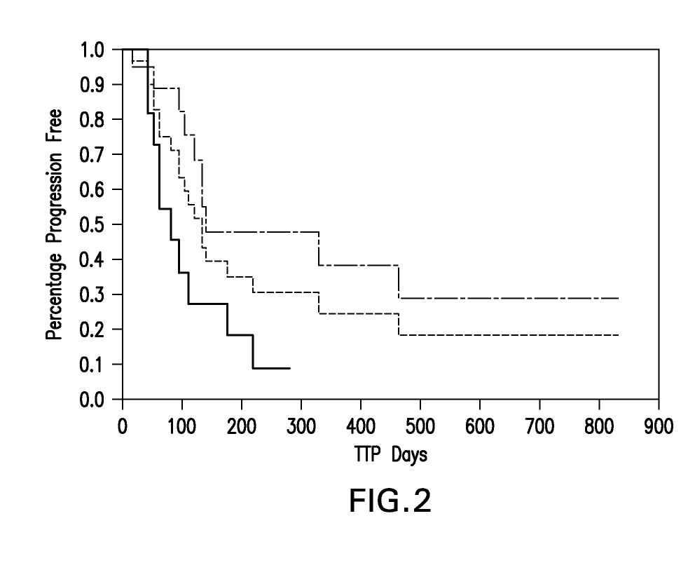

Three (10%) out of 29 patients with one post baseline CT scan achieved partial response (PR); two had non-small cell lung cancer (NSCLC) treated at 0.3 mg/kg/day and 10 mg/day respectively, and one had colorectal cancer (CRC) treated at 0.1 mg/kg/day (FIGS. 1 and 2a). An additional sixteen patients had stable disease lasting longer than 12 weeks, among which were patients with CRC (5), NSCLC (2), ovarian cancer (2), hepatocellular carcinoma (HCC) (2) and neuroendocrine tumour (2). Prolonged stable disease lasting more than 12 months with minimal toxicity was observed in four patients: alveolar soft part sarcoma (27 months), CRC (19 months), HCC (17 months), and renal cell carcinoma (18 months). The patient with CRC received 0.25 mg/kg/day of ABT-869 and developed cavitation of lung nodules, despite previous bevacizumab treatment, but required 2 dose reductions for grade 3 abdominal discomfort to 0.1 mg/kg. With a planned protocol deviation to allow increase in dose to 0.15 mg/kg/day, further tumor reduction and cavitation was observed after 11 months, suggestive of a dose response for antiangiogenic activity from ABT-869.

### L. Pharmacodynamic Analyses

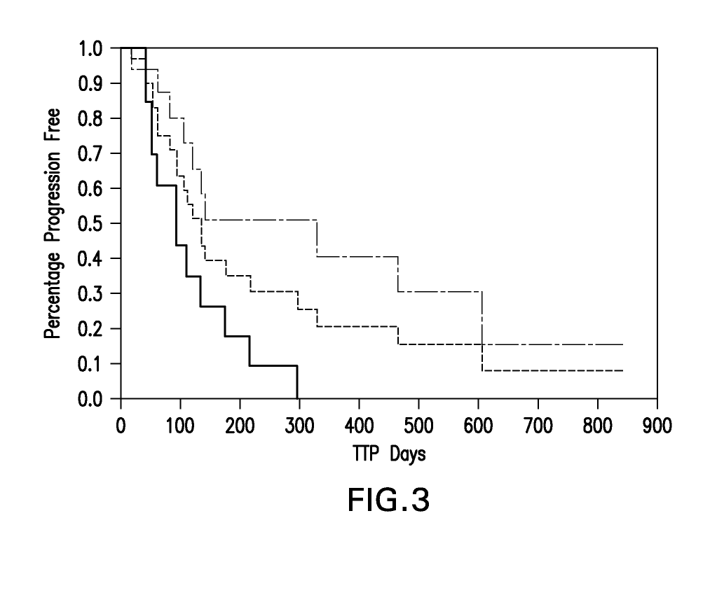

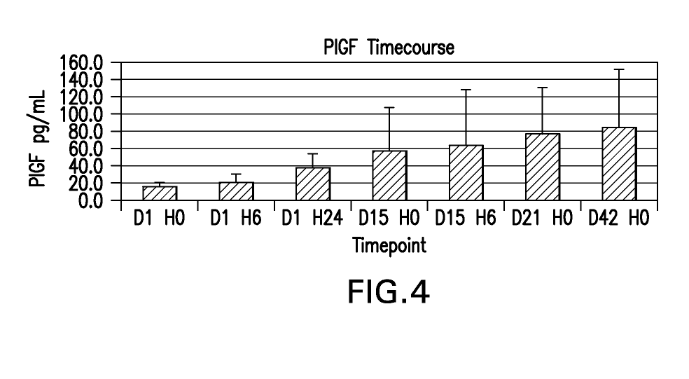

Human PlGF-1 (n=31) increased from 15.6±4.8 pg/mL at baseline to 20.7±9.4 pg/mL at 6 hours (p=0.02) and remained significantly elevated at 82.0±70.3 pg/mL on day 42 (p=0.0001) (See, FIGS. 1-4). Plasma VEGF (n=12) at baseline was 68.2±82.4 pg/mL and was significantly increased by day 15 at 124.8±80.7 pg/mL (p=0.004). Comparison of these biomarkers between 0.1 mg/kg/day and 0.25 mg/kg/day doses showed significantly higher human PlGF-1 (paired t-test p=0.017), percentage apoptotic CECs (p=0.028) and lower percentage activated CECs (p=0.027) on day 15 of treatment. In addition, percentage increase in PlGF-1 correlated positively with ABT869-AUCinf (r=0.57 p=0.0001) and ABT869-Cmax (r=0.42 p=0.0001).

### Example 2

Three phase 2 single agent multicenter clinical trials were run in hepatocellular carcinoma (HCC), renal cell carcinoma (RCC) and non-small cell lung cancer (NSCLC), each of which will be summarized separately.

HCC

An open-label, randomized, multicenter phase 2 trial (M06-879) of oral ABT-869 at 0.25 mg/kg daily in Child-Pugh A (hereinafter “C-PA”) or every other day (“QOD”) in Child-Pugh B (hereinafter referred to as “C-PB”) patients (hereinafter referred to as “pts”) until progressive disease (hereinafter referred to as “PD”) or intolerable toxicity, is ongoing. Key eligibility criteria included unresectable or metastatic HCC; up to one prior line of systemic treatment; and at least one measurable lesion by computed tomography (hereinafter “CT”) scan. The primary endpoint was the progression free (hereinafter “PF”) rate at 16 weeks. Secondary endpoints included objective response rate (hereinafter “ORR”), time to progression (hereinafter “TTP”), progression free survival (hereinafter “PFS”) and overall survival (hereinafter “OS”). CT scans were assessed centrally and by the investigators; presented results are from central assessment. Treatment will continue until disease progression or intolerable toxicity, or for up to 18 months after the last patient enrollment. Safety (graded by NCI CTCAE, Ver. 3.0) was evaluated at study visits

Inclusion criteria: Adults diagnosed with unresectable or metastatic HCC, measurable lesion by Response Evaluation Criteria In Solid Tumors (“RECIST”), received no more than 1 prior line of systemic treatment for HCC, had an ECOG performance status 0-2, no other malignancies within previous 5 years, and adequate organ function. Exclusion criteria: received anti-cancer therapy within 21 days (5 half-lives) before dosing or VEGF/PDGF/TKI therapy (prior Avastin allowed), untreated brain or meningeal metastases, Child-Pugh C hepatic impairment, proteinuria, uncontrolled hypertension, left ventricular Ejection Fraction <50%, >Grade 2 encephalopathy (CTC criteria), clinically significant uncontrolled conditions, and/or receiving therapeutic anticoagulation therapy or anti-retroviral therapy. The baseline characteristics and medical history of the patients are shown below in Table 4.

TABLE 4

| | All | | | | | |
|---|---|---|---|---|---|---|
| | Patients | | Child-Pugh A | | Child-Pugh B | |
| Baseline characteristics | n = 44 | | n = 38 | | n = 6 | |
| Median age, yrs (range) | 62.5 | (20-81) | 61.5 | (20-81) | 64.5 | (36-69) |
| Men, n (%) | 36 | (82) | 31 | (82) | 5 | (83) |
| Asian, n (%) | 39 | (89) | 33 | (87) | 6 | (100) |
| ECOG status, n (%) | | | | | | |
| 0 | 22 | (50) | 21 | (55) | 1 | (17) |
| 1 | 19 | (43) | 15 | (40) | 4 | (67) |
| 2 | 3 | (7) | 2 | (5) | 1 | (17) |
| Medical history at screening | | | | | | |
| Alcohol use, ever, n (%) | 22 | (50) | 22 | (58) | 1 | (17) |
| Hepatitis, n (%) | | | | | | |
| B | 27 | (61) | 23 | (61) | 4 | (67) |
| C | 4 | (9) | 4 | (11) | 0 | |
| Other, (%) | 13 | (30) | 11 | (29) | 2 | (33) |
| Extrahepatic spread, n (%) | 26 | (59) | 23 | (61) | 3 | (50) |
| Prior local regional therapies, n (%) | 24 | (55) | 20 | (53) | 4 | (67) |
| Prior systemic therapies, n (%) | | | | | | |
| 0 | 37 | (84) | 31 | (82) | 6 | (100) |
| 1 | 6 | (14) | 6 | (16) | 0 | |
| 2 | 1 | (2) | 1 | (3) | 0 | |

Results: 44 pts were enrolled from September 2007 to August 2008 at 6 centers internationally. There were 38 C-PA patients (median age, 63.5 y [range, 20-81]) and 6 C-PB patients (median age, 64.5 y [range, 36-69]) and 73.5% received no prior systemic therapy. 33/44 (75%) patients were followed for at least 16 weeks for disease progression.

For the 38 evaluable C-PA patients included in the per-protocol interim analysis, 13 (34.1%) were progression free at 16 weeks [95% CI 19.6, 51.4]. The estimated ORR was 7.9% [95% CI, 1.7, 21.4] for the 38 C-PA patients and 0% for the 6 C-PB patients who had at least one post-baseline CT scan reviewed by central imaging. For all 44 patients, median TTP was 5.4 months [95% CI, 3.7,-not reached] and median OS was 9.3 months [95% CI, 6.0, 11.0]. The most common adverse events (AEs) for all pts were hypertension (41%), fatigue (47%), diarrhea (38%), rash (35%), proteinuria (24%), vomiting (24%), cough (24%) and oedema peripheral (24%). The most common grade 3/4 AEs for all pts were hypertension (20.6%) and fatigue (11.8%). Most AEs were mild/moderate and reversible with interruption/dose reductions/or discontinuation of ABT-869.

Conclusions: ABT-869 appears to benefit HCC patients, with an estimated TTP of 112 days and an acceptable safety profile.

NSCLC

This ongoing, open-label, randomized, multicenter phase 2 trial of ABT-869 at 0.10 mg/kg daily (Arm A) and 0.25 mg/kg daily (Arm B) until progressive disease (PD) or intolerable toxicity, was initiated to assess antitumor activity and toxicity of ABT-869 in patients with NSCLC. Eligibility criteria included locally advanced or metastatic NSCLC; ≧1 prior systemic treatment, and >1 measurable lesion by RECIST criteria. The primary endpoint was the progression free (hereinafter “PF”) rate at 16 wks. Secondary endpoints were objective response rate (hereinafter “ORR”), time to progression (hereinafter “TTP”), progression free survival (hereinafter “PFS”) and overall survival (hereinafter “OS”). CT scans were assessed by the investigator and centrally; central assessment results are provided. Patients were randomized 1:1 to ABT-869, stratified by dose and Asian status. ABT-869 0.10 or 0.25 mg/kg was self-administered as a daily oral dose under fasting conditions. Patients receiving the 0.10 mg/kg dose, whose disease progressed, were allowed to cross-over to the 0.25 mg/kg dose within 30 days from the last 0.10 mg/kg dose. Safety (graded by NCI CTCAE, Ver. 3.0) was evaluated at study visits.

Inclusion criteria: Adults diagnosed with locally advanced or metastatic NSCLC, had at least 1 measurable non-radiated lesion by RECIST on CT scan, received at least 1 to 2 prior lines of systemic treatment for NSCLC, and no neo-adjuvant or adjuvant chemotherapy for NSCLC, ECOG performance status 0-2, no other active malignancy within the previous 5 years, adequate organ function. Exclusion criteria: received anti-cancer therapy within 21 days or 5 half-lives before dosing, radiation or major surgery within previous 21 days before dosing, or targeted VEGF/PDGF/TKI therapy (prior Avastin allowed), had untreated brain or meningeal metastases, history of >10% weight loss during 6 weeks before study entry, significant central thoracic lesions invading/abutting heart or major blood vessels, clinically relevant hemoptysis, proteinuria, symptomatic or persistent uncontrolled hypertension, left ventricular Ejection Fraction <50%, >Grade 2 encephalopathy (CTC criteria), had clinically significant uncontrolled conditions, was receiving therapeutic anticoagulation therapy or anti-retroviral therapy. The baseline characteristics and medical history of the patients are shown below in Table 5.

TABLE 5

| | | | 0.10 mg/kg | 0.25 mg/kg |
|---|---|---|---|---|
| | | All Patients | ABT-869 | ABT-869 |
| | | n = 139 | n = 65 | n = 74 |
| Baseline characteristics | | | | |
| Median age, yrs (range) | | 62 | 61 | 62 |
| Men, n (%) | | 82 (59.0) | 41 (63.1) | 41 (55.4) |
| Asian ethnicity, n (%) | | 49 (35.3) | 23 (35.4) | 26 (35.1) |
| ECOG status, n (%) | | | | |
| 0 | | 45 (33) | 23 (35) | 22 (30) |
| 1 | | 88 (63) | 38 (58) | 50 (67) |
| 2 | | 6 (4) | 4 (7) | 2 (3) |
| Medical history at screening | | | | |
| Smoker, n (%) | | 95 (68) | 41 (63) | 54 (73) |
| Non-smoker, n (%) | | 44 (32) | 24 (37) | 20 (27) |
| Squamous, n (%) | | 17 (12) | 8 (12) | 9 (12) |
| Non-squamous, n (%) | | 122 (88) | 57 (88) | 65 (88) |
| Prior systemic therapies, | | | | |
| n (%) | | | | |
| 1 | | 56 (40) | 25 (39) | 31 (42) |
| 2 | | 72 (52) | 34 (52) | 38 (51) |
| >2 | | 11 (8) | 6 (9) | 5 (7) |
| Endpoints | | | | |
| Primary | PFR at 16 Weeks | 48 (34.5) | 21 (32.3) | 27 (36.5) |
| | n (%) [95% CI] | [26.7, 43.1] | [21.2, 45.1] | [25.6, 48.5] |
| Secondary | PFS, median | 3.6 [3.0, 3.9] | 3.5 [2.0, 4.3] | 3.7 [3.1, 4.9] |
| | mo [95% CI] | | | |
| | OS, median | 9.0 [6.9, | 10.7 | 8.6 [5.6, |
| | mo [95% CI] | 12.8] | [6.9, —] | 12.8] |
| | Estimated ORR | 2 (1.4) | 0 | 2 (2.7) [0.3, |
| | n/N (%) [95% CI] | [1.7, 5.1] | | 9.4] |

PFR based on radiographic assessment by the central imaging center and on clinical assessment by the investigator
— not reached

Table 6 below shows the most Common Treatment-related Adverse Events

TABLE 6

| | | | | |
|---|---|---|---|---|
| | | 0.10 mg/kg | 0.25 mg//kg | |
| | All | ABT-869 | ABT-869 | |
| ABT-869-related | n = 139 | n = 65 | n = 74 | P-value |

Any grade, >20% of all patients

| | | | | |
|---|---|---|---|---|
| Diarrhea | 36 (25.9) | 10 (15.4) | 26 (35.1) | 0.011* |
| Nausea | 32 (23.0) | 11 (16.9) | 21 (28.4) | — |
| Fatigue | 55 (39.6) | 19 (29.2) | 36 (48.6) | 0.024* |
| Anorexia | 42 (30.2) | 13 (20.0) | 29 (39.2) | 0.016* |
| Proteinuria | 28 (20.1) | 5 (7.7) | 23 (31.1) | <0.001* |
| Hypertension | 47 (33.8) | 12 (18.5) | 35 (47.3) | <0.001* |

Grade 3 or 4, ≧10%
of all patients

| | | | | |
|---|---|---|---|---|
| Hypertension | 18 (12.9) | 1 (1.5) | 17 (23.0) | <0.001* |

P-value for comparison between the high vs. low dose groups using Fisher's Exact test
*Statistically significant difference

Results: 138 patients (pts) were enrolled from 08/07-10/08 from 27 centers 120/139 (86%) patients were followed for at least 16 weeks for disease progression. A total of 10 pts had squamous cell histology (all 10 were included in the interim analysis). The remainder had non-squamous histology. Median age was 64 years and 62 years in Arm A and B respectively. For the interim analysis population (Arm A, n=24; Arm B, n=24), 16 (33.3%) pts were PF at 16 wks: 7 (29.2%) in Arm A and 9 (37.5%) in Arm B. The ORR in Arm A (n=30) was 0% and 7.3% in Arm B (n=41). The median TTP and median PFS were 110 and 109 days, and 112 days and 108 days in Arm A and B, respectively. The most common adverse events (AEs) in Arm A were fatigue (35%), nausea (21%), and anorexia (21%), and in Arm B were hypertension (51%), fatigue (51%), diarrhea (43%), anorexia (41%), nausea (31%), proteinuria (31%) and vomiting (26%). The most common grade 3/4 toxicities in the Arm A were fatigue (7%), ascites (5%), dehydration (5%), pleural effusion (5%), and in the Arm B were hypertension (23%), fatigue (8%), PPE syndrome (8%), dyspnoea (6%) and stomatitis (6%). Most AE's were mild/moderate and reversible with interruptions/dose reduction/or discontinuation of ABT-869.

Conclusions: ABT-869 demonstrates an acceptable safety profile and appears to be active in NSCLC patients.

RCC

Phase II, single-arm, open-label, multicenter trial in adults with advanced RCC, previously treated with sunitinib. Oral dose of ABT-869 0.25 mg/kg every day (“QD”) was self-administered by patients, under fasting conditions. Treatment continued until disease progression or intolerable toxicity. Efficacy endpoints: primary: objective response rate (hereinafter “ORR”); secondary: progression-free survival (hereinafter “PFS”), overall survival (hereinafter “OS”), and progression-free rate (hereinafter “PFR”) at week 16.

Inclusion Criteria: adults diagnosed with locally recurrent or metastatic RCC, at least 1 unidimensionally measurable lesion by RECIST, previous nephrectomy, received >2 cycles (12 wks) of treatment with sunitinib for RCC and stopped therapy due to disease progression within 100 days before screening, ECOG performance status 0-1, no history of other active cancer within the previous 5 years, life expectancy of at least 4 months, adequate organ function. Exclusion Criteria: received anti-cancer therapy within 21 days (5 half-lives) before dosing, major surgery within 21 days before dosing, TKI therapy targeting VEGFR and/or PDGFR other than sunitinib or sorafenib (prior bevacizumab allowed), or anti-retroviral therapy for HIV, proteinuria, symptomatic or persistent uncontrolled hypertension, left ventricular ejection fraction <50%, known autoimmune disease with renal involvement, clinically significant uncontrolled conditions. Tumor response was assessed at 8-week intervals using RECIST. Safety was evaluated through physical examinations, laboratory tests, assessment of ECOG performance status, and AEs. AEs were graded using the National Center Institute Common Terminology Criteria for AEs, version 3.0.

53 patients enrolled in the study from August 2007 to October 2008, across 12 centers in the US and Canada47/53 (89%) patients either developed disease progression or were followed for at least 24 weeks for disease progression. Baseline characteristics for the patients is shown in Table 7.

TABLE 7

| Baseline Characteristics | All Patients, n = 53 | |
|---|---|---|
| Median age, yrs (range) | 61.0 | (40-80) |
| Men, n (%) | 42 | (79) |
| ECOG status 0, n (%) | 19 | (36) |
| ECOG status 1, n(%) | 34 | (64) |
| Histology, n (%) | | |
| Clear cell | 43 | (81) |
| Non-clear cell | 10 | (19) |
| Prior systemic therapies, n (%) | | |
| 1 | 26 | (49) |
| 2 | 17 | (32) |
| >2 | 10 | (19) |
| Prior therapies, n (%) | | |
| Sunitinib | 53 | (100) |
| Cytokine | 12 | (23) |
| Sorafenib | 10 | (19) |
| Temsirolimus | 2 | (4) |
| Bevacizumab | 9 | (17) |

| | |
|---|---|
| Best response (PR) to prior sunitinib, % | 13.2 |

Reasons for Treatment Discontinuation

30 patients due to Progressive Disease (PD) (clinical, radiographic, or AE related to PD)

7 patients due to AEs not related to PD

1 patient for other reasons

At the time of this analysis, 15 patients remained on

The response of the patients is shown in Table 8 and AEs are shown below in Table 9.

TABLE 8

| | | All Patients, n = 53 | |
|---|---|---|---|
| Endpoints | | Central Imaging | Site Assessment |
| Primary | ORR, % [95% CI] | 9.4 [3.1, 20.7] | 17.0 [8.1, 29.8] |
| Secondary | PFS Median | 5.4 [3.6, 6.3] | 5.8 [3.9, 7.3] |
| | mo [95% CI] | | |
| | PFR at 16 Weeks | 49.1 [35.1, 63.2] | 54.7 [40.4, 68.4] |
| | % [95% CI] | | |

| | |
|---|---|
| OS Median | 11.6 [10.1, —] |
| mo [95% CI] | |

— not reached

TABLE 9

| | |
|---|---|
| ABT-869-related | All patients (n = 53), n (%) |

Any grade, ≧20% of all patients

| | |
|---|---|
| Diarrhea | 37 (69.8) |
| Fatigue | 37 (69.8) |
| Hypertension | 30 (56.6) |
| Nausea | 22 (41.5) |
| Hand-foot skin | 18 (34.0) |
| reaction* | |
| Weight loss | 18 (34.0) |
| Anorexia | 17 (32.1) |
| Proteinuria | 17 (32.1) |
| Vomiting | 16 (30.2) |
| Mucosal inflammation | 11 (20.8) |
| Rash | 11 (20.8) |

Grade 3 or 4, ≧10% of all patients

| | |
|---|---|
| Hypertension | 15 (28.3) |
| Fatigue | 10 (18.9) |
| Diarrhea | 9 (17.0) |
| Hand-foot skin reaction* | 9 (17.0) |

*Coded as palmar-plantar erythrodysesthesia

Analysis of PlGF-1 concentrations in the above described three Phase 2 monotherapy studies was performed using the optimized PlGF-1 assay described in Example 3, with greater sensitivity which necessitated the identification of new efficacy and toxicity thresholds. A greater variation in baseline concentrations of PlGF-1 was noted in this larger patient population therefore analysis of the PlGF-1 was performed using values normalized by baseline subtraction (in which the pretreatment number was subtracted from the post treatment number and used to obtain the resulting value). The baseline values of PlGF-1 were determined to be predictive of response with those having PlGF-1 concentrations lower than 24 pg/mL ultimately performing better (Time to Progression (TTP) 168 versus 112 days, p=0.03). Changes in PlGF-1 following administration of ABT-869 were analyzed using either an early sample (Day 8 and/or 15), taken prior to any dose reduction/interruption or using a mean PlGF-1 AUC (the AUC was derived using all PlGF-1 values for a given patient during the dosing period including values derived during dose interruptions) to reflect the dose intensity over the first 3 weeks of treatment and resulted in the determination of optimized PlGF-1 efficacy thresholds. For Child-Pugh A patients, the early PlGF-1 threshold of 81.5 pg/mL was associated with improved overall survival (316 versus 266 days, p=0.03). This same PlGF-1 threshold was also predictive patients for improved median TTP (211 versus 58 days, p=0.05) and OS (352 versus 284 days, p=0.3) in a Phase 2 study of ABT-869 in renal cell carcinoma (RCC). The AUC threshold for the first 22 days similarly segregates patients for improved response (less than 66.5 pg/mL, OS=268 days and greater than 66.5 pg/mL the median was not reached, p=0.04).

Many of the patients on these studies experienced toxicities that necessitated either a dose interruption and/or a dose reduction. Using the early predose reduction samples, a clear PlGF-1 toxicity cutoff of 148 pg/mL above which 87% of the Hepatocellular Cancer (HCC) patients had “toxicity” (grade 3/4 events that result in a dose reduction/interruption, or grade 2 toxicities that result in dose interruptions) was identified. This threshold was predictive of toxicity in all 3 studies. Utilization of this toxicity threshold could play a role in proactive toxicity management. Interestingly, the patients with the best overall survival had high PlGF-1 values within the first two weeks of treatment and a subsequent dose reduction. Analysis of the mean AUC PlGF-1 concentration through day 56 in HCC and day 112 in RCC takes into account both the initial dose and the impact of dose reductions at timepoints relevant to a majority of patients for the respective studies. Application of the PlGF-1 efficacy threshold (generated from the mean AUC through day 22) of 66.5 pg/mL and the 148 pg/mL toxicity threshold to this data set demonstrated that an optimal PlGF-1 concentration could be identified, and resulted in increased OS in HCC (316 versus 193 days, p=0.015) and in RCC (median not determined versus 405 days, p=0.035).

### Example 3

Analysis of PlGF-1 concentrations in the above described three Phase 2 monotherapy studies in Example 2 were performed using a PlGF-1 assay as described in greater detail in Example 15 (referencing Example 11) of U.S. application Ser. No. 12/485,114 filed Jun. 16, 2009 (incorporated by reference for its teachings regarding same). Briefly, this assay employs an ARCHITECT® immunoassay format for detection of human PlGF-1, and utilizes anti-PlGF-1 monoclonal antibody from the 2-826-335 hybridoma cell line (MAB826) as the capture reagent immobilized on paramagnetic microparticles, and uses anti-PlGF-1 monoclonal antibody from the 1-255-713 or 1-255-2675 hybridoma cell lines (MAB255) as the conjugate reagent labeled with acridinium. The conjugate reagent was either an intact IgG monoclonal antibody, an F(ab′)2, or Fab fragment. The ARCHITECT® assay was run as described in Example 11 of U.S. application Ser. No. 12/485,114 using a range of sample volumes from 50 to 100 microliters for optimization of the assay. The optimum sample volume of the assay is 50 microliters. PlGF-1 purchased from R&D Systems was used as calibrator material for the assay. PlGF-1 was first diluted in a buffer matrix called ‘calibrator diluent’ (a buffer containing 2-(N-morpholino)ethanesulfonic acid (MES), other salt, a protein blocker and an antimicrobial) to obtain concentrated intermediate stock solutions. Calibrators were prepared gravimetrically on an analytical balance by diluting the intermediate stock with the calibrator diluent. Calibrators were prepared at PlGF-1 concentrations of 0, 10, 30, 60, 500, and 1,500 pg/mL (Cal A, Cal B, Cal C, Cal D, Cal E, and Cal F, respectively). Specimens were collected in either EDTA plasma or serum collection tubes.

The immunoassay was carried out by automated ARCHITECT® i2000 analyzer (Abbott Laboratories, Abbott Park, Ill.). Briefly, the assay involved the following steps:

1. Mixing 50 μL of human sample with 50 μL of microparticles coated with anti-human PlGF-1 antibody (namely, MAB826). Similarly, calibrator solutions can be used in this step in place of the human sample to prepare a standard curve and calibrate the assay.

2. Incubating the reaction mixture for approximately 18 minutes at a maintained ambient temperature of 33-38° C. The human PlGF-1 antigen in the sample bound to the anti-human PlGF-1 antibody on the microparticles.

3. Unbound human PlGF-1 was separated from magnet-detained microparticle and drained into waste. The microparticles were washed with a phosphate buffer.

4. Adding 50 μL of the detection antibody labeled with an acridinium ester (monoclonal antibody MAb 255) to the reaction mixture.

5. Incubating the reaction mixture for approximately 4 minutes at 33-38° C. The anti-human PlGF-1 antibody-acridinium molecule forms a sandwich with human PlGF-1 captured by antibody immobilized on the microparticle.

6. Washing the microparticles with a phosphate buffer.

7. Adding Pre-trigger (acid solution with hydrogen peroxide) and Trigger (basic solution) to cause the captured human PlGF-1 to emit light, which was measured by the instrument as Relative Light Units (RLUs). RLUs are the designation for the optical unit of measurement utilized on the ARCHITECT® systems. The ARCHITECT® optics system is essentially a photomultiplier tube (PMT) that performs photon counting on the light emitted by the chemiluminescent reaction. The amount of light generated by the chemiluminescent reaction is proportional to the amount of acridinium tracer present in the reaction mixture, and thereby allows quantitation of the sample analyte that is also proportional to the amount of acridinium remaining in the reaction mixture at the time the chemiluminescent reaction occurs. The term “Relative Light Units” comes from the relation of the photon counting to a certain amount of acridinium. Each optics module is calibrated with a set of acridinium standards. When the chemiluminescent reaction occurs, light is emitted and the photons are measured over a 3 second time period. The PMT converts the photons counted to digital signal, which is then sent to a circuit board for processing. The optics circuit board converts the digital signal from the PMT to an analog signal that is proportional to the photons counted, which is in turn proportional to the amount of acridinium present. This analog signal is then further processed to produce an RLU value. This relationship was established to produce a standard for calibration of the optics module, where the different acridinium standards have RLU values assigned to them. So, while the RLU unit itself is arbitrary, it is proportional (i.e., relative) to a certain amount of acridinium.

A range of sample volumes from 50 to 100 microliters was used during optimization of the assay. The preferred sample volume is 50 to 75 microliters and the optimal sample volume is 50 microliters. PlGF-1 purchased from R&D Systems was used as calibrator material for the assay. Calibrators are prepared at PlGF-1 concentrations of 0, 10, 30, 60, 500, and 1,500 pg/mL.

The preferred immunoassay format was used to measure PlGF-1 in 400 apparently normal individuals. The specimens were purchased from ProMedDx, LLC (Norton, Mass.) and comprised of 200 males and 200 females. The specimens were collected in either EDTA plasma or serum collection tubes. The results are shown in Table 10, where the median PlGF concentration is 16.0 pg/mL and the upper 97.5 percentile is 25.8 pg/mL. The lowest sample is 7.9 pg/mL and the highest value is 29.8 pg/mL in this sample set.

TABLE 10

| | |
|---|---|
| Sample size | 400 |
| Median | 16.0 |
| Lowest value | 7.9 |
| Highest value | 29.8 |
| Geometric mean | 16.1 |
| Kolmogorov-Smirnov test | accept Normality (P = 0.751) |
| for Normal distribution | |

Percentiles

| | |
|---|---|
| 0.5 | 9.7 |
| 2.5 | 10.7 |
| 5 | 11.1 |
| 25 | 13.9 |
| 75 | 18.6 |
| 95 | 23.6 |
| 97.5 | 25.8 |
| 99.5 | 28.0 |

Values back-transformed after logarithmic transformation.

The preferred immunoassay format was used to measure PlGF in pregnant individuals with gestational age ranging from 4.5 to 39 weeks. The specimens were collected in EDTA plasma. The results are shown in FIG. 5 (N=1,490 specimens), where a steady increase in PlGF value is observed with increasing gestational age up to approximately 30 weeks. After approximately 32 weeks, the PlGF values are widely scattered. The PlGF concentration in these specimens ranges from approximately 7.0 pg/mL to approximately 4,500 pg/mL. Specimens with initial values greater than 1,500 pg/mL were retested after a 4-fold dilution to provide a result within the calibration range.

These results show that the preferred immunoassay format is able to detect human PlGF-1 in pregnant individuals. Studies have also been done with the preferred immunoassay format in individuals with preeclampsia, patients with cardiac conditions, and patients with carcinoma such as renal cell carcinoma, hepatocellular carcinoma, and non small cell lung carcinoma.

One skilled in the art would readily appreciate that the present disclosure is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of monitoring whether a subject being administered a drug has obtained an efficacious blood level of said drug in order to optimize dosing or scheduling, the method comprising the steps of:
(a) contacting a test sample obtained from a subject being administered N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea with a first capture antibody that binds to human PlGF-1 or a human PlGF-1 fragment to form a first capture antibody-human PlGF-1 complex,
(b) contacting said first capture antibody-human PlGF-1 complex with a second antibody that binds to human PlGF-1 or human PlGF-1 fragment and that has been conjugated to a detectable label (“detection antibody”) to form a second capture antibody-human PlGF-1 detection complex;
(c) determining the amount of the second capture antibody-human PlGF-1 detection complex formed in step (b) by detecting the detectable label, wherein the amount of the second complex formed is the amount of human PlGF-1 or human PlGF-1 fragment contained in the test sample; and
(d) comparing the amount of human PlGF-1 or human PlGF-1 fragment in the test sample determined in step (c) with a predetermined level, wherein if the concentration of human PlGF-1 or human PlGF-1 fragment determined in step (c) is lower than the predetermined level, then the subject is considered not to be receiving an efficacious amount of N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea and further wherein, if the concentration of human PlGF-1 or human PlGF-1 fragment determined in step (c) is the same as or higher than the predetermined level, then the subject is considered to be receiving an efficacious amount of N-[4-(3-amino-1H-indazol-4-yl)phenyl]-N′-(2-fluoro-5-methylphenyl)urea or analog of N-[4-(3-amino-1H-indazol-4-yl)phenyl]-N′-(2-fluoro-5-methylphenyl)urea.

2. The method of claim 1, wherein the capture antibody is monoclonal antibody 264 and the detection antibody is polyclonal antibody pB264.

3. The method of claim 1, wherein the predetermined level when the capture antibody is a monoclonal antibody and the detection antibody is a polyclonal antibody is about 30 picograms per milliliter at about 24 hours after the subject first receives treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea.

4. The method of claim 1, wherein the predetermined level when the capture antibody is a monoclonal antibody and the detection antibody is a polyclonal antibody is about 40 picograms per milliliter to about 75 picograms per milliliter at about 15 days after the subject first receives treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea.

5. The method of claim 1, wherein the capture antibody is monoclonal antibody 826 and the detection antibody is monoclonal antibody 255.

6. The method of claim 1, wherein when the capture antibody is a monoclonal antibody and the detection antibody is a monoclonal antibody and the concentration of human PlGF-1 or human PlGF-1 fragment determined in step (c) is increased by about 60 picograms per milliliter to about 150 picograms per milliliter when compared to the predetermined level at the steady state about either 8 or 15 days after the subject first receives treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea.

7. The method of claim 1, wherein the subject is being treated for cancer selected from the group consisting of lung cancer, breast cancer, stomach cancer, bladder cancer, colon cancer, pancreatic cancer, ovarian cancer, prostate cancer, renal cancer, hepatocellular cancer, rectal cancer, hematopoietic malignancies, glioblastoma and infantile hemangioma.

8. The method of claim 2, wherein the dose or schedule for treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea is adjusted to place the patient in the range of about 40 picograms per milliliter to about 75 picograms per milliliter based on the comparison in step (d).

9. The method of claim 5, wherein the dose or schedule for treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea is adjusted to place the patient in the range of about 60 picograms per milliliter to about 150 picograms per milliliter based on the comparison in step (d).

10. The method of claim 1, wherein the method is adapted for use in an automated system or semi-automated system.

11. A method of monitoring a response of a subject receiving treatment for cancer with an anti-cancer drug, the method comprising the steps of:
(a) contacting a test sample obtained from a subject receiving treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea with a first capture antibody that binds to human PlGF-1 or human PlGF-1 fragment to form a first capture antibody-human PlGF-1 complex;
(b) contacting said first capture antibody-human PlGF-i complex with a second antibody that binds to human PlGF-1 and that has been conjugated to a detectable label (“detection antibody”) to form a second capture antibody-human PlGF-1 detection complex;
(c) determining the amount of the second capture antibody-human PlGF-1 detection complex formed in step (b) by detecting the detectable label, wherein the amount of the second complex formed is the amount of human PlGF-1 or human PlGF-1 contained in the test sample; and
(d) comparing the amount of human PlGF-1 or human PlGF-1 in the test sample determined in step (c) with a predetermined level, wherein if the concentration of human PlGF-1 or human PlGF-1 fragment determined in step (c) is lower than the predetermined level, then the subject is considered not to be responding to treatment with the N-[4-(3-amino-1H-indazol-4-yl)phenyl]-N′-(2-fluoro-5-methylphenyl)urea or an analog of N-[4-(3-amino-1 H-indazol-4-yl)phenyl]-N′-(2-fluoro-5-methylphenyl)urea and treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea is discontinued and further wherein, if the concentration of human PlGF-1 or human PlGF-1 fragment determined in step (c) is the same as or higher than the predetermined level, then the subject is considered to be responding to treatment with the N-[4-(3-amino-1H-indazol-4-yl)phenyl]-N′-(2-fluoro-5-methylphenyl)urea or analog of N-[4-(3-amino-1H-indazol-4-yl)phenyl]-N′-(2-fluoro-5-methylphenyl)urea.

12. The method of claim 11, wherein the capture antibody is monoclonal antibody 264 and the detection antibody is polyclonal antibody pB264.

13. The method of claim 11, wherein the capture antibody is monoclonal antibody 826 and the detection antibody is monoclonal antibody 255.

14. The method of claim 11, wherein the cancer is selected from the group consisting of lung cancer, breast cancer, stomach cancer, bladder cancer, colon cancer, pancreatic cancer, ovarian cancer, prostate cancer, renal cancer, hepatocellular cancer, rectal cancer, rectal cancer, hematopoietic malignancies, glioblastoma and infantile hemangioma.

15. The method of claim 11, wherein the method is adapted for use in an automated system or semi-automated system.

16. A method of determining whether a subject who is predisposed to a disease or who is suffering from a disease will benefit from receiving treatment with a drug, the method comprising the steps of:
(a) contacting a test sample obtained from a subject predisposed to a disease or suffering from at least one disease and administered N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea with a first capture antibody that binds to human PlGF-1 or human PlGF-1 fragment to form a first capture antibody-human PlGF-1 complex;
(b) contacting said first capture antibody-human PlGF-1 complex with a second antibody that binds to human PlGF-1 and that has been conjugated to a detectable label (“detection antibody”) to form a second capture antibody-human PlGF-1 detection complex;
(c) determining the amount of the second capture antibody-human PlGF-1 detection complex formed in step (b) by detecting the detectable label, wherein the amount of the second complex formed is the amount of human PlGF-1 or human PlGF-1 contained in the test sample; and
(d) comparing the amount of human PlGF-1 or human PlGF-1 in the test sample determined in step (c) with a predetermined level, wherein if the concentration of human PlGF-1 or human PlGF-1 fragment determined in step (c) is lower than the predetermined level, then a determination is made that the subject will not benefit from receiving further or continued treatment with the N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea and further wherein, if the concentration of human PlGF-1 or human PlGF-1 fragment determined in step (c) is the same as or higher than the predetermined level, then a determination is made that the subject will benefit from receiving further or continued treatment with the N-[4-(3-amino-1H-indazol-4-yl)phenyl]-N′-(2-fluoro-5-methylphenyl)urea or analog of N-[4-(3-amino-1H-indazol-4-yl)phenyl]-N′-(2-fluoro-5-methylphenyl)urea.

17. The method of claim 16, wherein the capture antibody is monoclonal antibody 264 and the detection antibody is polyclonal antibody pB264.

18. The method of claim 16, wherein the at least one disease is cancer selected from the group consisting of lung cancer, breast cancer, stomach cancer, bladder cancer, colon cancer, pancreatic cancer, ovarian cancer, prostate cancer, renal cancer, hepatocellular cancer, rectal cancer, rectal cancer, hematopoietic malignancies, glioblastoma and infantile hemangioma.

19. The method of claim 16, wherein the method is adapted for use in an automated system or semi-automated system.

20. A method of treating a subject suffering from at least one cancer selected from the group consisting of lung cancer, breast cancer, stomach cancer, bladder cancer, colon cancer, pancreatic cancer, ovarian cancer, prostate cancer, renal cancer, hepatocellular cancer, rectal cancer, hematopoietic malignancies, glioblastoma and infantile hemangioma, the method comprising the steps of:
(a) obtaining a test sample from the subject suffering from cancer and who is receiving treatment with a predetermined amount of N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea;
(b) contacting the test sample with a first capture antibody that binds to human PlGF-1 or a human PlGF-1 fragment to form a first capture antibody-human PlGF-1 complex,
(c) contacting said first capture antibody-human PlGF-1 complex with a second antibody that binds to human PlGF-1 or human PlGF-1 fragment and that has been conjugated to a detectable label (“detection antibody”) to form a second capture antibody-human PlGF-1 detection complex;
(d) determining the amount of the second capture antibody-human PlGF-1 detection complex formed in step (c) by detecting the detectable label, wherein the amount of the second complex formed is the amount of human PlGF-1 or human PlGF-1 fragment contained in the test sample;
(e) comparing the amount of human PlGF-1 or human PlGF-1 fragment in the test sample determined in step (d) with a predetermined level; and
(f) treating a subject having a concentration of human PlGF-1 or human PlGF-1 fragment determined in step (d) that is lower than the predetermined level with: (i) an adjusted amount of N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea that is higher than the predetermined amount of N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea recited in step (a); (ii) a drug other N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea; or (iii) combinations of (i) and (ii).

21. The method of claim 20, wherein the capture antibody is monoclonal antibody 264 and the detection antibody is polyclonal antibody pB264.

22. The method of claim 20, wherein the predetermined level when the capture antibody is a monoclonal antibody and the detection antibody is a polyclonal antibody is about 30 picograms per milliliter at about 24 hours after the subject first receives treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea.

23. The method of claim 20, wherein the predetermined level when the capture antibody is a monoclonal antibody and the detection antibody is a polyclonal antibody assay is about 40 picograms per milliliter to about 75 picograms per milliliter at about 15 days after the subject first receives treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea.

24. The method of claim 20, wherein the capture antibody is monoclonal antibody 826 and the detection antibody is monoclonal antibody 255.

25. The method of claim 20, wherein when the capture antibody is a monoclonal antibody and the detection antibody is a monoclonal antibody and the concentration of human PlGF-1 or human PlGF-1 fragment determined in step (e) is increased by about 60 picograms per milliliter to about 150 picograms per milliliter when compared to the predetermined level at the steady state about either 8 or 15 days after the subject first receives treatment with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea.

26. A kit comprising:
(a) at least one antibody selected from the group consisting of: monoclonal antibody 264, polyclonal antibody pB264 and combinations thereof; and
(b) instructions for using said kit for a purpose selected from the group consisting of: determining whether a subject receiving treatment with a drug has obtained an efficacious blood level of said drug, wherein the drug is N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea; determining whether a subject receiving treatment with a drug has obtained an efficacious blood level of said drug, wherein the drug is N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, monitoring a response of a subject receiving treatment for cancer with an anti-cancer drug, wherein the drug is N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, confirming biological activity of N-[4-(3-amino 1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea in a subject being administered N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea and combinations thereof.

27. A kit comprising:
(a) at least one antibody selected from the group consisting of: monoclonal antibody 826, monoclonal antibody 255 and combinations thereof; and
(b) instructions for using said kit for a purpose selected from the group consisting of: determining whether a subject who is predisposed to a disease or who is suffering from a disease will respond to treatment with a drug, wherein the drug is N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, determining whether a subject who is predisposed to a disease or who is suffering from a disease will respond to treatment with a drug, wherein the drug is N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, monitoring progression of disease in a subject being treated with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, monitoring progression of disease in a subject being treated with N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea, confirming biological activity of N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea in a subject being administered N-[4-(3-amino-1H-indazol-4-yl)phenyl]-N′-(2-fluoro-5-methylphenyl)urea or an analog of N-[4-(3-amino-1H-indazol-4-yl)phenyl]-N′-(2-fluoro-5-methylphenyl)urea and combinations thereof.
